SEQUENCE LISTING (12 sequences)

SEQ ID NO: 1 - protein, 20 aa, Synthetic construct
ERLVYISFRQGLLTDTGLSL

SEQ ID NO: 2 - protein, 20 aa, Synthetic construct
SSPFLSKRSLSRSPIPSRKS

SEQ ID NO: 3 - protein, 20 aa, Synthetic construct
LSRSPIPSRKSMKSRSRSPA

SEQ ID NO: 4 - protein, 20 aa, Synthetic construct
LMTKDAPDSLSPENYGNFDT

SEQ ID NO: 5 - protein, 20 aa, Synthetic construct
RTTGQMVAPSLSPNKKMSSE

SEQ ID NO: 6 - protein, 20 aa, Synthetic construct
GILARNLVPMVATVQGQNLK

SEQ ID NO: 7 - protein, 25 aa, Synthetic construct
AFFRNHPATWKNTIRHNLSLHKCFV

SEQ ID NO: 8 - protein, 25 aa, Synthetic construct
RGRVLKKFKSDSRLAQRRFIRGWGL

SEQ ID NO: 9 - protein, 25 aa, Synthetic construct
EEKDSESHLAEDHHAVSTEAEDRSY

SEQ ID NO: 10 - protein, 9 aa, Synthetic construct
HPATWKNTI

SEQ ID NO: 11 - protein, 9 aa, Synthetic construct
SRLAQRRFI

SEQ ID NO: 12 - protein, 9 aa, Synthetic construct
HLAEDHHAV

(19) United States
(12) Patent Application Publication
(10) Pub. No.: US 2023/0390370 A1
(43) Pub. Date: Dec. 7, 2023

(54) NEOANTIGEN VACCINES FOR PANCREATIC CANCER

(71) Applicants: William Gillanders, St. Louis, MO (US); William Hawkins, St. Louis, MO (US); Simon Goedegebuure, St. Louis, MO (US); Hui Peng, St. Louis, MO (US); Lijin Li, St. Louis, MO (US)

(72) Inventors: William Gillanders, St. Louis, MO (US); William Hawkins, St. Louis, MO (US); Simon Goedegebuure, St. Louis, MO (US); Hui Peng, St. Louis, MO (US); Lijin Li, St. Louis, MO (US)

(73) Assignee: Washington University, St. Louis, MO (US)

(21) Appl. No.: 18/328,209

(22) Filed: Jun. 2, 2023

Related U.S. Application Data
(60) Provisional application No. 63/348,998, filed on Jun. 3, 2022.

(51) Int. Cl. A61K 39/00; A61P 35/00; A61K 39/395

(52) U.S. Cl. CPC A61K 39/001152; A61P 35/00; A61K 39/3955; A61K 39/39566; A61K 39/0011; A61K 2039/53; A61K 2039/852; A61K 2039/585

(57) ABSTRACT

The present disclosure is directed to compositions and methods for treating pancreatic cancer. A method of treating pancreatic cancer includes administering a therapeutically effective amount of a composition including a neoantigen vaccine including at least one pancreatic cancer-associated neoantigen and at least one immune checkpoint inhibitor. The methods and compositions of the present disclosure are particularly useful for inducing a neoantigen-specific CD4 or CD8 T cell response against a tumor.

### CROSS-REFERENCE TO RELATED APPLICATIONS

This application claims priority from U.S. Provisional Application Ser. No. 63/348,998 filed on 3 Jun. 2022, which is incorporated herein by reference in its entirety.

### STATEMENT REGARDING FEDERALLY SPONSORED RESEARCH & DEVELOPMENT

This invention was made with government support under CA196510 awarded by the National Institutes of Health. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name “020217-US-NP_Sequence_Listing.xml” created on 1 Jun. 2023; 11,395 bytes). The subject matter of the Sequence Listing is incorporated herein by reference in its entirety.

### FIELD OF THE DISCLOSURE

The field of the disclosure relates generally to neoantigen vaccines. More specifically, the disclosure relates to neoantigen vaccine compositions and methods for treatment of pancreatic cancer.

### BACKGROUND OF THE DISCLOSURE

Cancer neoantigens are important targets of cancer immunotherapy and neoantigen vaccines are currently in development in pancreatic ductal adenocarcinoma (PDAC) and other cancer types. Immune regulatory mechanisms in pancreatic cancer may limit the efficacy of neoantigen vaccines. Accordingly, there is a need for pancreatic cancer treatment compositions and methods that improve the efficacy of neoantigen vaccines.

### BRIEF DESCRIPTION OF THE DISCLOSURE

An aspect of the present disclosure provides for a method of treating pancreatic cancer in a subject, the method comprising administering a therapeutically effective amount of a composition comprising a neoantigen vaccine comprising at least one pancreatic cancer-associated neoantigen and at least one immune checkpoint inhibitor. In some embodiments, the at least one immune checkpoint inhibitor comprises at least one of a PD-1 inhibitor, a PD-1L inhibitor, and a TIGIT inhibitor. In some embodiments, the at least one immune checkpoint inhibitor comprises a PD-1 inhibitor and a TIGIT inhibitor. In some embodiments, administering the therapeutically effective amount of the composition increases survival, enhances T cell antitumor immune response or infiltration, or reduces tumor volume in the subject compared to administering a neoantigen vaccine or checkpoint inhibitor alone. In some embodiments, the least one immune checkpoint inhibitor comprises at least one of an anti-PD1 antibody, an anti-PDL1 antibody, and an anti-TIGIT antibody. In some embodiments, the at least one pancreatic cancer-associated neoantigen is identified based on at least one of exome sequencing and RNA sequencing of a pancreatic tumor or cancer cell. In some embodiments, the at least one pancreatic cancer-associated neoantigen comprises at least a portion of a protein or peptide encoded by a gene selected from the group consisting of CAR12, CDK12, FOXP3, FAM129C, and ANK2. In some embodiments, the at least one pancreatic cancer-associated neoantigen comprises at least one amino acid sequence, each amino acid sequence at least 95% identical to a sequence selected from the group consisting of SEQ ID NOS: 1-5. In some embodiments, the at least one pancreatic cancer-associated neoantigen comprises at least one amino acid sequence, each amino acid sequence at least 95% identical to SEQ ID NO: 1 or SEQ ID NO: 2. In some embodiments, the at least one pancreatic cancer-associated neoantigen comprises at least one amino acid sequence, each amino acid sequence at least 95% identical to a sequence selected from the group consisting of SEQ ID NOS: 7-12. In some embodiments, the therapeutically effective amount of the composition induces a neoantigen-specific CD4 or CD8 T cell antitumor response. In some embodiments, the therapeutically effective amount of the composition increases the number of functional tumor-specific CD4 T cells in a tumor microenvironment (TME) or spleen of the subject compared to administering a neoantigen vaccine or checkpoint inhibitor alone. In some embodiments, the therapeutically effective amount of the composition reduces or prevents TIGIT-mediated exhaustion of neoantigen-specific T cells compared to administering a neoantigen vaccine or checkpoint inhibitor alone.

Another aspect of the present disclosure provides for a pharmaceutical composition comprising a neoantigen vaccine, the neoantigen vaccine comprising at least one pancreatic cancer-associated neoantigen and at least one immune checkpoint inhibitor. In some embodiments, the at least one pancreatic cancer-associated neoantigen is derived from at least a portion of a protein or peptide encoded by a gene selected from the group consisting of CAR12, CDK12, FOXP3, FAM129C, and ANK2. In some embodiments, the at least one pancreatic cancer-associated neoantigen comprises at least one amino acid sequence, each amino acid sequence at least 95% identical to a sequence selected from the group consisting of SEQ ID NOS: 1-5. In some embodiments, the at least one pancreatic cancer-associated neoantigen comprises at least one amino acid sequence, each amino acid sequence at least 95% identical to a sequence selected from the group consisting of SEQ ID NOS: 7-12. In some embodiments, the at least one immune checkpoint inhibitor comprises at least one of a PD-1 inhibitor, a PD-1L inhibitor, and a TIGIT inhibitor. In some embodiments, the at least one immune checkpoint inhibitor comprises a PD-1 inhibitor and a TIGIT inhibitor.

Yet another aspect of the present disclosure provides for a vaccine comprising a peptide comprising at least one pancreatic cancer-associated neoantigen amino acid sequence, wherein each pancreatic cancer-associated neoantigen amino acid sequence is at least 95% identical to a sequence selected from the group consisting of SEQ ID NOS: 1-5 and SEQ ID NOS: 7-12; and a pharmaceutically acceptable carrier or adjuvant.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The embodiments described herein may be better understood by referring to the following description in conjunction with the accompanying drawings.

FIG. 1(A-B) is an exemplary embodiment of immunogenic KPC4580P neoantigens mCAR12 and mCDK12 in accordance with the present disclosure. FIG. 1A shows a schematic experimental design. C57BL/6 mice (n=5 each group) were vaccinated twice on day 0 and day 7. Five days later (day 12), spleens were harvested and single-cell suspensions were prepared for ex vivo IFN-γ ELISpot. FIG. 1B shows ELISpot assay results indicated that peptide vaccination with mCAR12 and mCDK12 was able to induce neoantigen-specific immune response in vivo. Adjuvant (Poly IC) alone, or peptide pools (100 μg each) used as vaccines were indicated above each plot. Each symbol represents data from an individual animal.

FIG. 2(A-C) is an exemplary embodiment of mCAR12/mCDK12 neoantigen vaccine eliciting T cell responses in accordance with the present disclosure. FIG. 2A shows an experimental schedule. C57BL/6 mice were vaccinated three times with a mix of 100 pg each mCAR12 and CDK12, or with Poly IC alone (n=3 each group). 5 days after the final vaccination, spleen cells were prepared and stimulated ex vivo with mCAR12 and mCDK12 peptides. FIG. 2B shows peptide-specific IFN-γ and TNF-α production was measured by intracellular cytokine staining and flow cytometry. FIG. 2C shows CD3+ T splenocytes from vaccinated mice were used in an IFN-γ ELISpot assay with or without the addition of MHC class II or MHC class I-blocking antibody. Numbers of spots in response to 20-mer mCAR12 or mCDK12 peptides were shown.

FIG. 3(A-D) is an exemplary embodiment of neoantigen SLP vaccine inducing CD4 and CD8 T cell responses and inhibiting pancreatic cancer growth in accordance with the present disclosure. FIG. 3A shows an experimental schema. Mice were inoculated with KPC4580P cells followed by vaccination with a neoantigen vaccine incorporating mCAR12/mCDK12 peptides+poly IC (Vac, n=11) or poly IC alone (Poly IC, n=8) at the indicated time points. FIG. 3B shows tumor volumes measured twice a week over time. Individual and mean±SEM of tumor sizes were plotted. **P<0.01, t-test. FIG. 3C shows neoantigen-specific CD4 and CD8 T cells analyzed 22 days after tumor inoculation. Spleen cells were stimulated ex vivo with peptides corresponding to mCAR12/mCDK12, and analyzed by intracellular cytokine staining for IFN-γ and TNF-α. Representative dot plots and summary data are also shown. FIG. 3D shows granzyme B expression on CD44⁺ splenic CD4 and CD8 T cells examined at day 22 after tumor inoculation. Data in FIG. 3C-FIG. 3D were presented as mean SEM (n=6-7). *P<0.05, **P<0.01, ***P<0.001, t-test.

FIG. 4(A-E) is an exemplary embodiment of tumor regression induced by neoantigen vaccine requiring both CD4 and CD8 T cells in accordance with the present disclosure. FIG. 4A shows an experimental schema of T cell deletion. Anti-CD4 or anti-CD8 depleting antibodies were administered (i.p.) before vaccination and throughout the study (n=7 or 8 in each group). FIG. 4B shows tumor volume measured over time after KPC4580P cells inoculation. FIG. 4C shows an experimental schema of T cell adoptive transfer study. KPC4580P tumor-bearing mice were vaccinated with neoantigen mCDK12/mCAR12 or with poly IC alone as indicated. At day 35, CD3⁺ T cells were isolated from spleens and were adoptively transfer (4×10⁶ T cells per recipient mouse) into immunocompromised Rag-1^{-/-} mice followed by tumor challenge one day later (n=6 to 7 each group). FIG. 4D shows IFNγ- and GzmB—producing T cells were detected in donor spleens by intracellular cytokine staining after in vitro stimulation with mCAR12/mCDK12 peptides. FIG. 4E shows KPC4580P tumor growth in Rag-1^{-/-} mice received T cell adoptive transfer.

FIG. 5(A-D) is an exemplary embodiment of neoantigen SLP vaccine enhancing effector CD4 and CD8 T cells and decreasing suppressor CD4 T cells in the tumor microenvironment in accordance with the present disclosure. FIG. 5A shows flow cytometry analyses of TILs at day 22 after KPC4580P inoculation revealed that treatment with neoantigen SLP vaccine (Vac) is associated with more tumor-infiltrating CD4 and CD8 T cells than control treatment (Poly IC). Percentage of CD4 and CD8 T cells among CD45+ cells and the total cell number per mg tumor are shown. FIG. 5B shows GzmB expression on CD4 and CD8 TILs at day 22. FIG. 5C shows flow cytometry analysis of CD11a and CD49d among CD4 TIL performed at day 22. FIG. 5D shows Foxp3⁺CD25⁺ Treg and TIGIT⁺Foxp3⁺ CD4 T cells were detected in CD4 TIL at day 22. Significance was determined using t-test (n=3; mean±SEM; *P<0.05; **P<0.01). The experiment was repeated once and similar results were obtained.

FIG. 6(A-C) is an exemplary embodiment of CD49d^{hi}CD11a^{hi} surrogate markers identifying a CD4 effector T cell subpopulation in KPC4580P tumor bearing mice in accordance with the present disclosure. FIG. 6A shows a representative gating strategy used in flow cytometry data analysis to identify the CD4 and CD8 TILs. FIG. 6B shows representative plots showing CD11a, CD49d and IFN-γ staining on CD4 T cells after ex vivo re-stimulation with mCAR12/mCDK12 peptides. Spleen cells were harvested at day 22 from KPC4580P tumor-bearing mice vaccinated with neoantigens mCAR12/mCDK12 or Poly IC alone. FIG. 6C shows collective data showing frequencies of IFN-7-producing cells by CD49d^{hi}CD11a^{hi} and CD49d^{lo}CD11a^{lo} CD4 T cells after stimulation with mCAR12/mCDK12.

FIG. 7(A-C) is an exemplary embodiment of TIGIT expression in T cells increasing during tumor development in accordance with the present disclosure. FIG. 7A shows the mean fluorescence indexes (MFI) indicate the expression levels of PD-L1, CD155 and MHC class II on cultured KPC4580P cells with or without IFN-γ treatment for 24 h. Percentages of MHC II+ cells were also shown. FIG. 7B shows flow cytometric analysis of TIGIT expression in CD44- and CD44⁺ CD4 T cells in spleens from KPC4580P tumor bearing mice at days 15, 22, and 29 after tumor injection. Bar graph summarizes data from 3-4 animals generated at each time point. FIG. 7C shows percentage of TIGIT⁺ cells among CD44⁺ CD4 and CD8 T cells from the spleens (SP) and TIL of KPC4580P tumor bearing mice at day 22. *P<0.05 and **P<0.01, student t-test.

FIG. 8(A-C) is an exemplary embodiment of a significant percentage of neoantigen-specific CD4 T cells expressing high levels of TIGIT in accordance with the present disclosure. FIG. 8A and FIG. 8B show splenocytes from vaccinated KPC4580P tumor-bearing mice stimulated ex vivo with mCAR12/mCDK12 and stained with the surface markers TIGIT, PD1, CD49d, CD11a, and intracellular cytokine IFN-7. FIG. 8A shows CD4⁺ T cells were gated based on the expression of surrogate markers CD49d and CD11a. Around 24% of the neoantigen-specific CD4 T cells (CD49d^{hi}CD11a^{hi}) are TIGIT⁺, compared to less than 1% of the CD49d^{lo}CD11a^{lo} naïve CD4 T cells. FIG. 8B shows percentages of PD-1, IFN-g, or CD226 expressing cells were compared between the TIGIT⁺ and TIGIT⁻ populations (CD49d^{hi}CD11a^{hi} CD4 cells). FIG. 8C shows splenocytes from vaccinated mice were stimulated with a mixture of both mCAR12/mCDK12 peptides for 3 days in the presence of IL-2+/−anti-TIGIT Ab and rested for 3 days. On day 6, cells were re-stimulated with a mixture of both mCAR12/mCDK12 peptides for intracellular cytokine staining. Each symbol represents data derived from an individual animal (n=3; mean±SEM).*P<0.05; **P<0.01; ***P<0.001, Student t-test. Data in FIG. 8A-FIG. 8C were generated in a single experiment. Similar results were obtained in two additional experiments.

FIG. 9(A-D) is an exemplary embodiment of PD-1/PD-L1 blockade upregulating TIGIT expression on T cells in accordance with the present disclosure. FIG. 9A shows a treatment timeline for KPC4580P-bearing mice. Three days following KPC4580P implantation, mice received Vac (100 μg each mCDK12 and mCAR12). Anti-PD-1 antibody (200 μg) was administered twice a week as shown starting at day 10. FIG. 9B shows tumor volumes measured every 3 to 4 days. Student's t-test was performed using measurements collected at day 34. *P<0.05, **P<0.01. FIG. 9C and FIG. 9D show flow cytometry analysis of TIGIT expression on CD4 T cells (FIG. 9C) and CD8 T cells (FIG. 9D) from the spleens of KPC4580P tumor bearing mice at day 22. Unpaired t-test, *P<0.05, **P<0.01.

FIG. 10(A-G) is an exemplary embodiment of combination PD1/TIGIT blockade enhancing the response to neoantigen SLP vaccine in accordance with the present disclosure. FIG. 10A shows a treatment timeline for KPC4580P-bearing mice. Three days following KPC4580P implantation, mice were vaccinated with neoantigen SLP and received treatment of anti-TIGIT antibody and anti-PD1 antibody, as indicated. FIG. 10B shows tumor volumes (mm³) were measured twice a week, starting at day 9. Individual tumor growth data can be found in FIG. 11A. *P<0.05 at day 34, Student's t-test. FIG. 10C shows Kaplan-Meier curves showing animal survival rates in each treatment group. *P=0.0139, Log-rank (Mantel-Cox) test, comparing all 4 groups. FIG. 10D and FIG. 10E show spleen cells were stimulated ex vivo with a mixture of mCAR12/mCDK12 peptides and were analyzed by flow cytometry for the expression of cell surface markers and intracellular molecules. Percentage of populations of cells among gated CD4⁺ (FIG. 10D) or CD8+(FIG. 10E) T cells were shown. FIG. 10F shows tumor-infiltrating CD4 and CD8 T cells were assessed by flow cytometry. Percentages of CD45+ cells that are CD4+ and CD8+ are shown. Absolute CD4 and CD8 T cell count per mg tumor can be found in FIG. 11B. FIG. 10G shows tumor-infiltrating T cells were harvested and stained for Treg and the surface expression of the exhaustion markers TIGIT and PD-1. Quantitation of CD4⁺ Treg cells and PD-1⁺ CD8 T cells as percentages of total tumor-infiltrating CD4 and CD8 T cells, respectively, are shown. Absolute CD25⁺Foxp3⁺ Treg number per mg tumor mass can be found in FIG. 11B. *P<0.05, **P<0.01, ***P<0.001, unpaired Student's t-test. Representative data from one of three experiments with similar results were shown.

FIG. 11(A-C) is an exemplary embodiment of PD-1/TIGIT dual blockade enhancing the response to neoantigen SLP vaccine in accordance with the present disclosure. FIG. 11A shows individual KPC4580P tumor growth data corresponding to FIG. 10B. Mice were inoculated with KPC4580P tumor cells. Three days later, tumor-bearing mice were vaccinated with neoantigen SLP followed by anti-TIGIT and anti-PD-1 antibody treatment, as indicated. Individual tumor sizes (mm³) were measured twice a week. FIG. 11B shows the number of CD4 and CD8 T cells per mg of KPC4580P tumors at day 22 after indicated treatments. FIG. 11C shows CD4eff/Tregand CD8/Tregratios of TILs in the KPC4580P tumors after indicated treatments. Each symbol indicates data from an individual animal. Ordinary one-way ANOVA multiple comparisons were performed for statistical significance, ** P<0.01, *** P<0.001, **** P<0.0001.

FIG. 12(A-C) is an exemplary embodiment of TIGIT restraining T cell responses in human PDAC in accordance with the present disclosure. FIG. 12A shows CyTOF analysis of TIGIT expression in human CD4 and CD8 T cells in the PBMCs from PDAC patients (n=12) and healthy donors (HD, n=8). FIG. 12B shows CyTOF analysis of TIGIT expression in human CD4 and CD8 T cells isolated from tumors (n=10) and uninvolved tissues (n=2) of PDAC patients. Each dot represents data from an individual human subject. Data were presented as Mean±SEM. *P<0.05, Mann-Whitney tests. FIG. 12C shows PBMCs from a PDAC patient vaccinated with neoantigen DNA vaccine were cultured with a mix of 3 neopeptides (FFA) plus IL-2 for 3 days with or without the anti-TIGIT antibody. The cells were rested for 3 days followed by FFA re-stimulation and analyzed by intracellular cytokine staining and flow cytometry. A similar two-fold increase in IFN-γ producing CD4 and CD8 T cells was obtained when anti-TIGIT antibody was added to the culture of PBMCs from the same patient that was re-stimulated with the viral CEF peptide pool (data not shown).

### DETAILED DESCRIPTION OF THE DISCLOSURE

Combination TIGIT/PD1 blockade enhances the efficacy of neoantigen vaccines in a model of pancreatic cancer. As disclosed herein, targeting immune checkpoint signaling pathways in pancreatic ductal adenocarcinoma (PDAC) improves the efficacy of neoantigen vaccines.

An established model of PDAC was used (KPC4580P) to test whether neoantigen vaccines generate therapeutic efficacy against PDAC. Two immunogenic neoantigens were focused on, resulting from mutations in the CAR12 and CDK12 genes. A neoantigen vaccine was tested containing two 20-mer synthetic long peptides and poly IC, a TLR agonist. The ability of neoantigen vaccine alone, or in combination with PD-1 and/or TIGIT signaling blockade was investigated to impact tumor growth. The impact of TIGIT signaling on T cell responses in human PDAC was also assessed.

Neoantigen vaccines induce neoantigen-specific T cell responses in tumor-bearing mice and slow KPC4580P tumor growth. However, KPC4580P tumors express high levels of PD-L1 and the TIGIT ligand, CD155. A subset of neoantigen-specific T cells in KPC4580P tumors are dysfunctional, and express high levels of TIGIT. PD1 and TIGIT signaling blockade in vivo reverses T cell dysfunction and enhances neoantigen vaccine-induced T cell responses and tumor regression. In human translational studies, TIGIT signaling blockade in vitro reverses neoantigen-specific T cell dysfunction following vaccination.

Taken together, preclinical and human translational studies support testing neoantigen vaccines in combination with therapies targeting the PD-1 and TIGIT signaling pathways in patients with PDAC.

As used herein, “antigen” or “neoantigen” refers to a portion or fragment of a molecule that is recognized by components of the immune system, such as a T cell, particularly when presented in the context of an MHC molecule, B cells, and antibodies. The antigen of a protein, such as a tumor antigen, preferably comprises a continuous or discontinuous portion of said protein and preferably has a length of 5 to 30. In certain aspects, an antigen may comprise a contiguous sequence and may be 9, 10, 11, 12, 13, 14, 15, 16, 17, 18, 19, 20, 21, 22, 23, 24, or 25 amino acids in length. In certain aspects, an antigen may comprise a contiguous sequence and may be at least 20, 21, 22, 23, 24 or 25 amino acids in length.

The at least one pancreatic cancer-associated neoantigen of the present disclosure may be from any protein expressed by a pancreatic cancer cell or tumor cell. In certain aspects, the at least one pancreatic cancer-associated neoantigen is identified based on exome sequencing and/or RNA sequencing of a pancreatic tumor or cancer cell. In certain aspects, the at least one pancreatic cancer-associated neoantigen comprises an amino acid sequence at least 95% identical to a sequence selected from the group consisting of SEQ ID NOS: 1-5 or SEQ ID NOS: 7-12. In certain aspects, the at least one pancreatic cancer-associated neoantigen is derived from a protein or peptide encoded by a gene selected from the group consisting of CAR12, CDK12, FOXP3, FAM129C, and ANK2.

One aspect of the disclosure is a method of treating pancreatic cancer in a subject, the method comprising administering a therapeutically effective amount of a composition comprising a neoantigen vaccine comprising at least one pancreatic cancer-associated neoantigen and at least one immune checkpoint inhibitor

In methods of the disclosure, the dose administered to a subject in a method of the invention can be any dose suitable for treating pancreatic cancer. In conjunction with the present disclosure, those skilled in the art are capable of identifying a dose appropriate for the chosen formulation and method of delivery.

In methods of the disclosure, therapeutic compositions, including vaccine compositions, of the invention may be administered by any route suitable for the subject being treated. Such routes of administration include, but are not limited to, injection, including parenteral administration, intravenous, intraperitoneal, intramuscular, and subcutaneous injection, oral administration, transmucosal administration, transdermal administration, topical administration, nasal administration, or ocular administration.

It is known in the art that cancers may be “staged” using a numerical scale that ranges from zero to four, with higher numbers indicating progressively larger and more invasive cancers. In pancreatic cancer treatment methods of the disclosure, the pancreatic cancer may be at any stage. In certain aspects, the pancreatic cancer may be Stage 0. In certain aspects, the pancreatic cancer may be Stage 1. In certain aspects, the pancreatic cancer may be Stage 2. In certain aspects, the pancreatic cancer may be Stage 3. In certain aspects, the pancreatic cancer may be Stage 4. Methods of staging pancreatic cancer are known to those skilled in the art.

In pancreatic cancer treatment methods of the disclosure, the therapeutic compositions, including vaccine compositions, of the disclosure may be administered prior to or following pancreatic cancer tumor removal. In certain aspects, the therapeutic compositions, including vaccine compositions, of the disclosure may be administered prior to or following neoadjuvant therapy. Examples of such neoadjuvant therapies include, but are not limited to chemotherapy, hormone therapy, and radiation therapy.

In pancreatic cancer treatment methods of the disclosure, a composition comprising at least one immune checkpoint inhibitor is administered. In some embodiments, the immune checkpoint inhibitor is a PD-1 inhibitor or a PD-1L inhibitor. A PD-1 inhibitor may be, for example, an anti-PD1 antibody, such as Pembrolizumab (Keytruda), Nivolumab (Opdivo), or Cemiplimab (Libtayo). A PD-1L inhibitor may be, for example, an anti-PD-1L antibody, such as Atezolizumab (Tecentriq), Avelumab (Bavencio), or Durvalumab (Imfinzi). In some embodiments, the immune checkpoint inhibitor is a TIGIT inhibitor, such as an anti-TIGIT antibody. For example, an anti-TIGIT antibody can be Vibostolimab, Domvanalimab, M6223, Ociperlimab, EOS884448, Etigilimab, or Tiragolumab. In some embodiments, the composition comprises more than one immune checkpoint inhibitor. In some preferred embodiments, the composition comprises a PD-1 inhibitor and a TIGIT inhibitor.

### EXAMPLES

### Combination TIGIT/PD-1 Blockade Enhances the Efficacy of Neoantigen Vaccines in a Model of Pancreatic Cancer

Introduction

PDAC is currently one of the deadliest cancers and is expected to become the second-leading cause of cancer-related death by 2030. Major factors responsible for the poor prognosis in PDAC include the resistance to both chemotherapy and immunotherapy, and the fact that many patients are diagnosed at an advanced stage, or with metastatic disease. In terms of cancer immunotherapy, PDAC presents unique therapeutic challenges due to a dense stroma and immunosuppressive tumor microenvironment (TME).

To date, immunotherapy in PDAC has been largely unsuccessful, including immune checkpoint inhibitors targeting PD-1/CTLA4. Recent studies suggest that the treatment efficacy of PD-1 blockade may depend on the presence of high-quality cancer neoantigens, i.e. antigens derived from genetic alterations present in the cancer genome. Based on these observations, specific attempts to vaccinate PDAC patients using neoantigen vaccines are under investigation. Initial clinical trials of neoantigen vaccines in melanoma and glioblastoma have been encouraging. There are significant conceptual advantages to targeting cancer neoantigens. The exclusive expression of neoantigens in tumors minimizes the risk of autoimmunity. Neoantigens are expressed exclusively in tumor cells, thereby minimizing the risk of autoimmunity. Neoantigen vaccines can be used to specifically target genetic alterations in cancer driver genes and/or broaden the profile of tumor-specific T cell responses. Nearly all PDAC tumors reportedly express potentially targetable neoantigens. Thus, targeting neoantigens through active vaccination holds promise as a novel immunotherapy in pancreatic cancer.

High-dimensional profiling of the immune landscape in PDAC demonstrates a deeply immune suppressive microenvironment. The majority of intratumoral CD8 T cells express a dysfunctional phenotype with elevated surface expression of exhaustion markers. TIGIT is a co-inhibitory receptor expressed on CD4, CD8, and NK cells, with PDAC cells expressing multiple TIGIT ligands such as CD155 and nectins 1 and 4 and TIGIT is one of the most common exhaustion markers expressed by intratumoral CD8 T cells. TIGIT blockade was found to restore T cell function in preclinical models, in particular when combined with PD-1/PD-L1 blockade. Restoring T cell function is dependent on the expression of the co-stimulatory receptor, CD226, which competes with TIGIT for binding to CD155. Elevated expression of CD155 was found in murine and approximately 80% of human PDAC and immune evasion was maintained by using the CD155/TIGIT pathway. Therefore, to combat an immunosuppressive pancreatic cancer TME, a combinatorial strategy is described herein comprising (1) neoantigen vaccination to generate neoantigen-specific immune responses, and (2) immune checkpoint blockade of TIGIT/PD-1.

The genetically engineered Kras^{G12D/+} Trp53^{R172H/+} p48-Cre (KPC) mouse model, was used herein. This model recapitulates important aspects of human PDAC, and is commonly used to study human pancreatic cancer. Cancer neoantigens have been demonstrated to play an important role in this model. The KPC4580P cell line derived from a spontaneous tumor in a KPC mouse has been studied. Irreversible electroporation can serve as an in situ vaccine to generate neoantigen-specific T-cell responses. Candidate neoantigens identified in KPC4580P were specifically targeted using a neoantigen vaccine, and the therapeutic efficacy of combination immunotherapy with TIGIT/PD-1 blockade was assessed.

Materials and Methods

Cell lines. KPC4580P cell line, derived from a spontaneous tumor in a male LSL-Kras^{G12D/+}; LSL-Trp53^{R172H/+}; Pdx1^{Cre/+}; LSL-Rosa26^{Luc/+} (KPC-Luc) mouse, were kindly provided by J. J. Yeh (University of North Carolina at Chapel Hill). KPC4580P cells were cultured in DMEM-F12 medium (Gibco) supplemented with 10% FBS, 2 mM L-glutamine, 1× penicillin/streptomycin (Gibco) at 37° C., and 5% CO2. The cell line was tested negative for mycoplasma.

Synthetic peptides. Peptides (20-mer) containing non-synonymous single nucleotide variants were synthesized by GenScript (Piscataway, NJ) and LifeTein (Somerset, NJ). The peptide sequences (N-C) for the preclinical KPC4580P model are as follows: mCAR12(15), ERLVYISFRQGLLTDTGLSL (SEQ ID NO: 1); mCDK12(15), SSPFLSKRSLSRSPIPSRKS (SEQ ID NO: 2); mCDK12(6), LSRSPIPSRKSMKSRSRSPA (SEQ ID NO: 3); mHOOK2(6), LMTKDAPDSLSPENYGNFDT (SEQ ID NO: 4); mHPS1(15), RTTGQMVAPSLSPNKKMSSE (SEQ ID NO: 5); and the control CMV peptide, GILARNLVPMVATVQGQNLK (SEQ ID NO: 6). Numbers in the parentheses indicate the positions of the mutated amino acids which are also underlined and in bold. In the rest of this manuscript, mCAR12(15) and mCDK12(15) are simply referred to as mCAR12 and mCDK12, respectively.

For the PDAC patient, the three immunogenic peptides are: FOXP3 (p.A439T),

(SEQ ID NO: 7)
AFFRNHPATWKNTIRHNLSLHKCFV;

### FAM129C (p.G520R),

(SEQ ID NO: 8)
RGRVLKKFKSDSRLAQRRFIRGWGL;

### ANK2 (p.R2714H),

(SEQ ID NO: 9)
EEKDSESHLAEDHHAVSTEAEDRSY.

The predicted minimal epitopes with highest affinity for corresponding HLA alleles are underlined and listed here as HPATWKNTI (SEQ ID NO: 10), SRLAQRRFI (SEQ ID NO: 11), and HLAEDHHAV (SEQ ID NO: 12).

Animals and reagents. All animal experiments were approved by the Institutional Animal Care and Use Committee (IACUC) of Washington University in St Louis. Wild-type (WT) C57BL/6 and Rag-1 knockout mice were purchased from The Jackson Laboratories. Rat anti-mouse PD-1 (clone RPM1-14), Rat anti-mouse TIGIT (clone 1G9), MHC class I (clone AF6-88.5.5.3) and class II antibody (clone M5/114), rat anti-mouse CD8 (clone 2.43), and rat anti-mouse CD4 (clone GK1.5) monoclonal antibodies were purchased from Bio X Cell.

Mouse models. For immunogenicity studies of mutated peptides, age-matched C57BL/6 mice were vaccinated once a week for 2-3 times. The readout was performed five days after the last immunization (see also Enzyme-linked ImmunoSpot and Flow cytometric analysis method sections). Vaccination was performed by subcutaneous (s.c.) injection of 100 μg synthetic peptides and 50 μg Poly IC formulated in PBS (100 μl total volume), with Poly IC alone as a negative control. For therapeutic tumor experiments, male C57BL/6 mice were inoculated s.c. with 5×10⁵ KPC4580P cells into the flank and randomly assigned to treatment groups. Mice were vaccinated (s.c.) at the tail base on days 3, 6, 10, 17, and 24. Tumor volume was measured with a caliper and calculated according to the formula (length×width²)/2. Mice were then sacrificed at the indicated time points or when the estimated tumor volume reached >2 cm³ (endpoint) or when a tumor is ulcerated.

In some experiments, repeated doses (250 μg per mouse i.p.) of anti-CD8 Ab or anti-CD4 Ab were administered to deplete CD8 or CD4 T cells. Successful depletion was confirmed by flow cytometry using PBMC or spleen cells. The depletion was maintained by administering the depleting antibody intraperitoneal once a week until the end of the study. Peptide vaccination was performed on these mice as described above. In some experiments, repeated 200 μg/dose of anti-PD1 Ab and 100 μg/dose of anti-TIGIT Ab were administered to mice (i.p.) twice a week.

Adoptive T cell transfer experiment. Subcutaneous pancreatic tumors were established by implanting 5×10⁵ KPC4580P cells in the right flank of male C57BL/6 mice. Neoantigen-vaccinated and Poly IC-treated mice were sacrificed at day 35 after tumor inoculation. The splenocytes were isolated and CD3 T cells were purified with the EasySep™ mouse T cell isolation Kit (StemCell). A total of 4×10⁶ CD3 T cells were adoptively transferred into each recipient Rag-1^{-/-} mouse through i.v. injection. One day later, 5×10⁵ KPC4580P tumor cells were implanted (s.c.) to the right flank of the recipient Rag-1^{-/-} mice. Tumor volume was measured with a caliper twice a week.

Enzyme-linked ImmunoSpot (ELISpot). After peptide immunization, splenocytes were cultured with or without peptides (4 μg/ml each mCAR12 and mCDK12) overnight at 37° C. in pre-coated 96-well plates (Mabtech), and cytokine secretion was detected with an anti-IFN-γ antibody (1 g/ml, clone R4-6A2, Mabtech). Subtyping of T-cell responses was performed with an MHC class II blocking antibody. All samples were tested in duplicates or triplicates.

Flow cytometry analysis. Splenocytes were stimulated with peptides (4 μg/ml each mCAR12 and mCDK12) and anti-CD28 (1 g/ml, BioLegend). Splenocytes treated with anti-CD3/CD28 served as a positive control. After incubation at 37° C. for 2 h, 1 l/ml of monensin (BioLegend) was added to each sample and incubated at 37° C. for an additional 5 h and then held at 4° C. overnight. The next day, cells were first stained with live/dead dye followed by staining with appropriate fluorescent antibody cocktails (CD3, CD4, CD8, CD44, CD11a, CD49d, TIGIT, CD226, PD-1) for 30 min on ice. Cells then were permeabilized and fixed using Foxp3 Cytofix/Cytoperm Buffer Set (eBioscience). Thereafter, cells were stained for IFN-γ, TNF-α, and Granzyme B (GzmB) (BD Biosciences). The samples were washed and resuspended in 250 μl of cold PBS containing 2% FBS for analysis using flow cytometry (BD Fortessa X-20 or BD FACScan). Fluorophore conjugated anti-mouse antibodies (clone names in parentheses) used in this study include: from BioLegend, CD11a (M17/4), CD3 (17A2), CD4 (GK1.5), CD4 (RM4-5), CD25 (3C7), CD44 (IM7), CD45 (30-F11), CD49d (R1-2), CD155 (TX56), CD226 (DNAM-1), GzmB (QA16A02), IFN-g (XMG1.2), TNF-a (MP6-XT22), PD-L1 (10F.9G2), TIGIT (1G9); from BD Biosciences, CD8a (53-6.7); from eBioscience, PD-1 (J43); and from Invitrogen, Foxp3 (FJK-16s). Anti-human antibodies used include: from BioLegend, CD3 (UCHT1), CD8 (RPA-T8), CD11a (HI111), IFN-g (4S.B3); form eBioscience, CD4 (OKT4); and from BD Biosciences, CD4 (SK3), IFN-g (B27). Flow cytometry data were analyzed using Flowjo 10 (TreeStar).

To study the tumors, mice were euthanized at day 22 post tumor injection. Portions of harvested tumors were processed using the Mouse Tumor Dissociation Kit (Miltenyi Biotec). The cells were passed through a 70-mm strainer to make single-cell suspensions. Cells were stained with live/dead dye followed by staining with proper antibody cocktails for 30 min on ice. Intracellular FoxP3 and GzmB staining was performed according to the manufacturer's protocol (Foxp3 Buffer Set, eBioscience). The samples were washed and resuspended in 250 μl of cold PBS containing 2% FBS for analysis using flow cytometry (BD Fortessa X-20). Data were analyzed using FlowJo v10 software.

Patient samples. PBMCs and tumor tissues were collected from pancreatic cancer patients between May 2018 and February 2020 using the Tissue Core funded by the Washington University SPORE in Pancreas Cancer in the Department of Surgery. The patients were diagnosed with resectable PDAC and treated with surgery as the initial treatment modality. Tissue and peripheral blood were collected at the time of surgery. All patients provided informed consent. The study conformed to the principles of the Declaration of Helsinki. The tissue acquisition protocol was approved by the Institutional Review Board at Washington University School of Medicine. For in vitro re-stimulation study using PBMCs from a PDAC patient treated with a polyepitope neoantigen DNA vaccine, 3×10⁵ PBMCs per well were cultured in a 96-well U-bottom plate for three days with 2 μM of each of the three neopeptides (FOXP3, FAM129C, and ANK2, see above) in the presence of recombinant human IL-2 (25 U/ml), anti-CD28 (1 μg/ml, clone CD28.2, BioLegend) and with or without anti-TIGIT antibody (10 μg/ml, clone A15153A, BioLegend). The cells were washed and rested in complete medium supplemented with 2.5 U/ml IL-2 for another three days. The cells were washed again and restimulated with the peptide pool (2 μM each) and anti-CD28 (1 μg/ml) for 5 h. Brefeldin-A (GolgiPlug, BD Biosciences) was added for the final 4 h. The cells were harvested and stained for cell surface markers and intracellular cytokines before being analyzed by flow cytometry.

Cytometry by Time of Flight (CyTOF). Cryopreserved PBMC were thawed in a 37° C. water bath and washed in pre-warmed cell culture medium (RPMI-1640, 10% FCS, 1×L-glutamine, and 1×penicillin/streptomycin supplemented with 1:10,000 benzonase (Sigma-Aldrich). Cells were then rested in complete medium for 1 hour at 37° C. before staining. PBMC (3×10⁶) were first stained with 5 mM cisplatin (Sigma) for 3 minutes on ice. After blocking with 50 μg/mL of human IgG (BD Biosciences) for 5 minutes, cells were stained with a master mix of titrated amounts of metal-labeled antibodies at 4° C. for 45 minutes. Surface-stained cells were permeabilized and fixed using FOXP3/Transcription Factor Staining Buffer (ThermoFisher) for 45 minutes on ice. After washing in permeabilization buffer (ThermoFisher), cells were then incubated for intracellular staining with a titrated panel of antibodies in permeabilization buffer for 45 minutes on ice. After washing in CytoPBS, cells were stained with 62.5 nM Iridium nucleic acid intercalator (Fluidigm) diluted in 2% paraformaldehyde (Electron Microscopy Sciences) in PBS overnight at 4° C. Finally, the cells were washed once with PBS, once with MilliQ water, and then diluted in water containing 10% EQ Calibration Beads (Fluidigm) before acquisition on a CyTOF2 mass cytometer (Fluidigm). Following this, the data were normalized using the normalization beads. The data were analyzed using the Cytobank online software.

Statistical analysis. GraphPad Prism 8 software was used for all statistical analyses. All data were presented as means±standard error (SEM). Intergroup comparisons were performed using a two-tailed unpaired t-test, and P<0.05 was considered statistically significant. Survival benefit was determined using log-rank test (Mantel-Cox). *P<0.05, **P<0.01, ***P<0.001.

Results

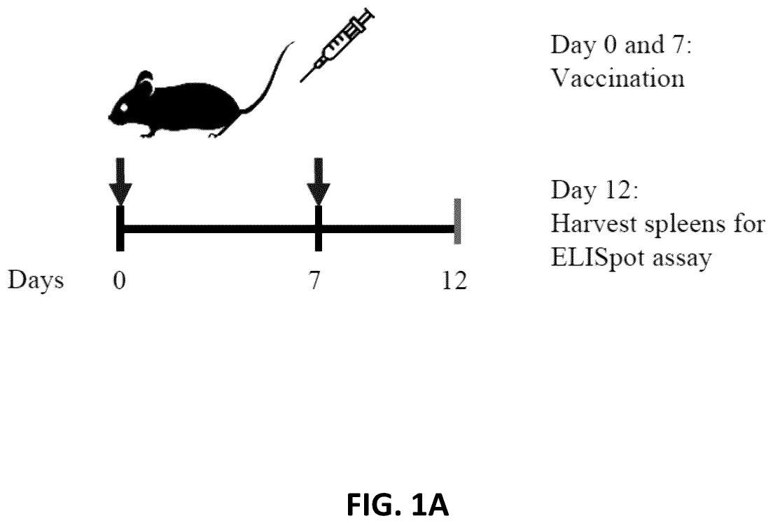

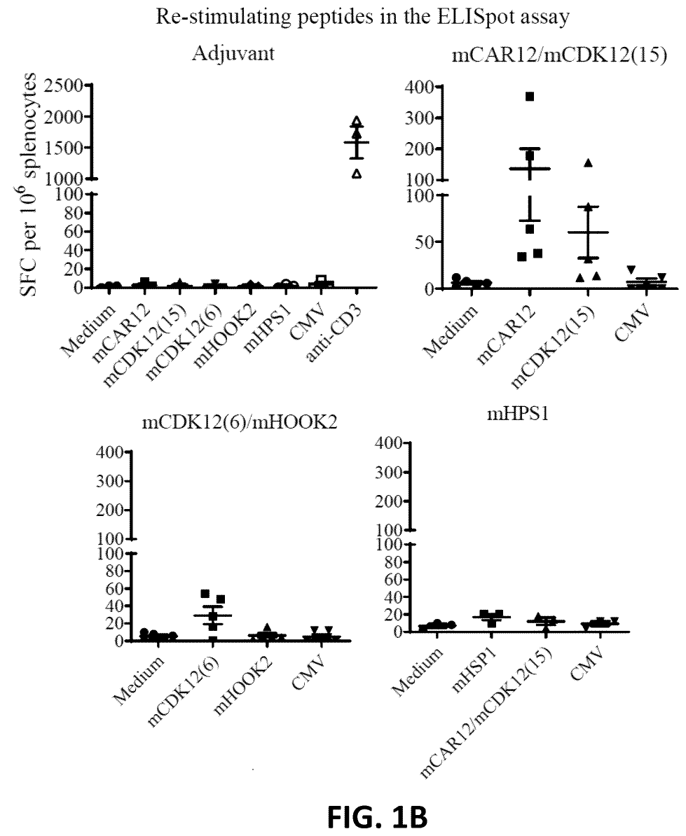

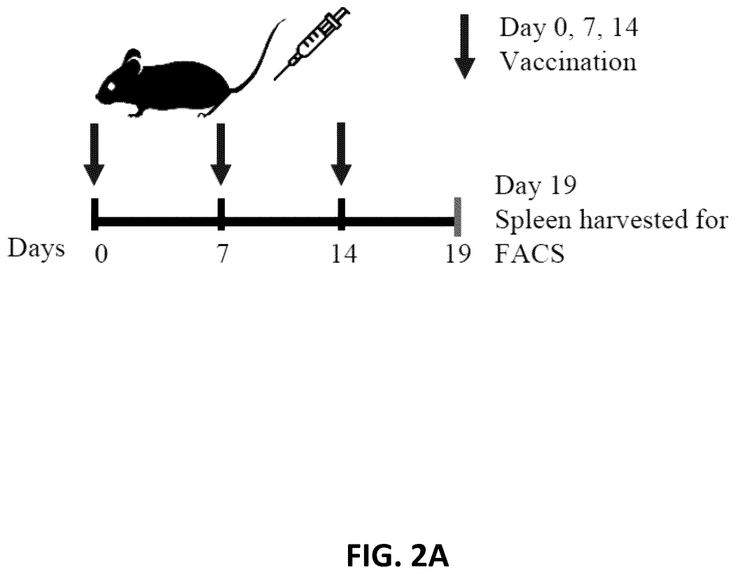

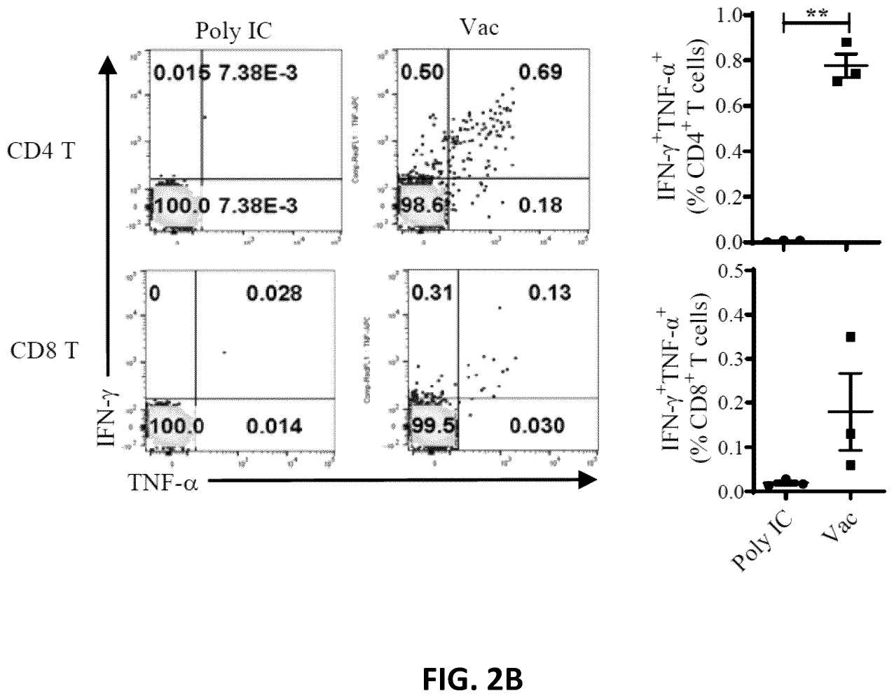

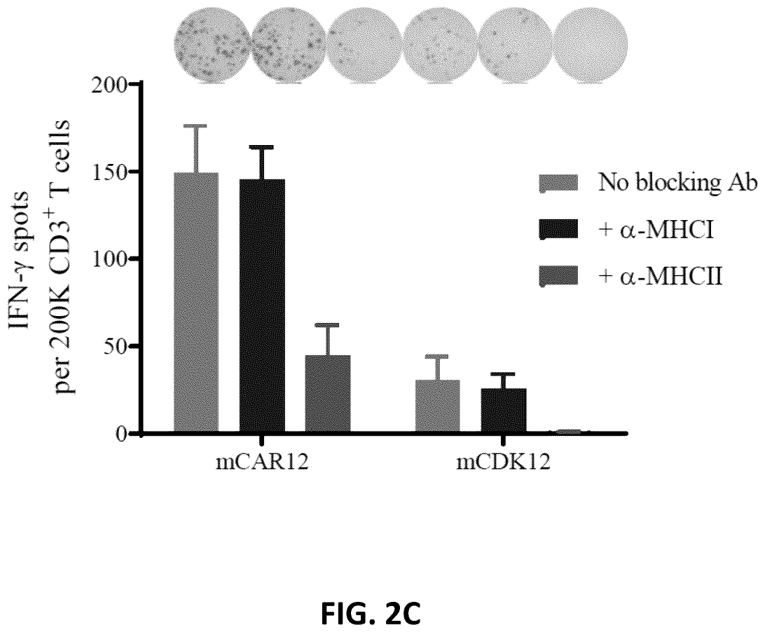

Credentialing cancer neoantigens in the KPC4580P pancreatic cancer model. The subcutaneous KPC4580P pancreatic cancer model was studied, which has a similar mutation burden as human PDAC. It was previously demonstrated that irreversible electroporation (IRE) of KPC4580P tumor induces complete regression in a subset of tumor-bearing animals and the antitumor responses were CD4/CD8 T cell-dependent. Whole-exome sequencing and RNA sequencing (RNA-seq) were performed to identify KPC4580P neoantigens. ELISpot assay results showed that IRE and vaccination with irradiated tumor cells were able to generate T cell reactivity against five peptides. To determine the potential of targeting these cancer neoantigens with vaccine therapy, naïve C57BL/6 mice were vaccinated using synthetic long peptides (SLP). The amino acid sequences of these five SLPs (mCAR12, mCDK12(15), mCDK12(6), mHOOK2, and mHSP1) are listed in the Materials and Methods. Vaccination with two of the neoantigen SLPs, namely mCAR12 and mCDK12, were able to generate a response above the background seen in mice vaccinated with adjuvant poly IC alone (FIG. 1A and FIG. 1). Further analysis of T cells revealed that multifunctional (IFN-γ+/TNF-α+) neoantigen-specific T cells were detected only in mice vaccinated with mCAR12/mCDK12 neoantigens and not in the control animals (FIG. 2A and FIG. 2B). Both mCAR12 and mCDK12 peptides induced predominantly CD4 T cell responses, as the addition of anti-NMC class II antibody complete blocked reactivity to mCDK12 and significantly decreased the number of IFN-γ secreting cells specific to mCAR12 (FIG. 2C). mCAR12 also stimulates CD8 T cell responses, albeit less robustly compared to CD4 T cell responses. It was concluded that mCDK12 and mCAR12 are immunogenic neoantigens for PKC4580P tumor model and the present disclosure describes neoantigen vaccines incorporating these two neoantigens.

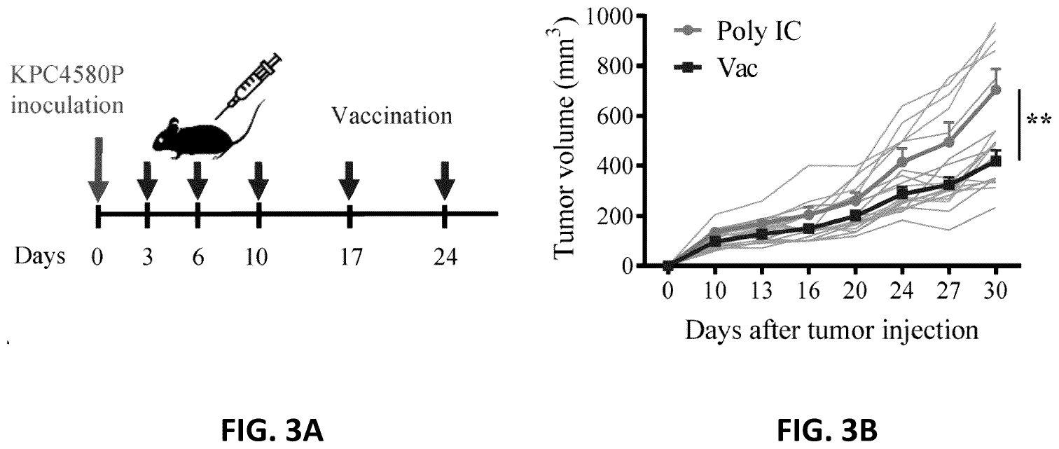

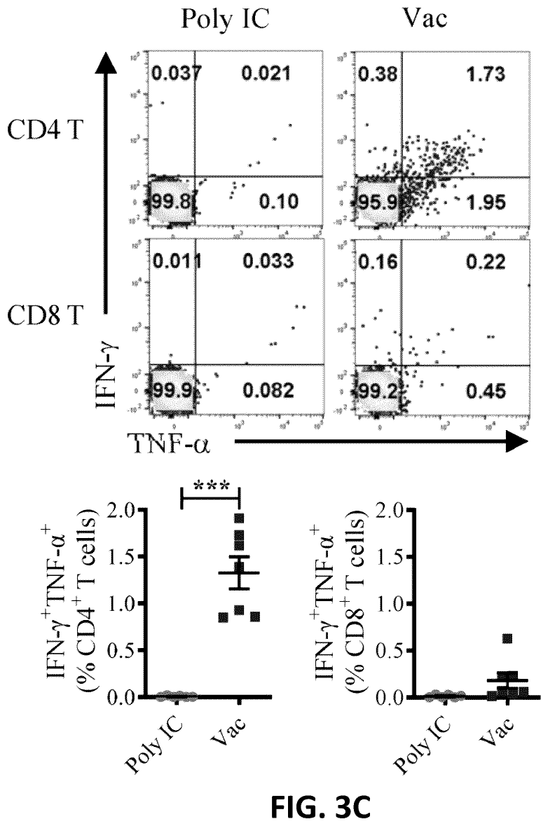

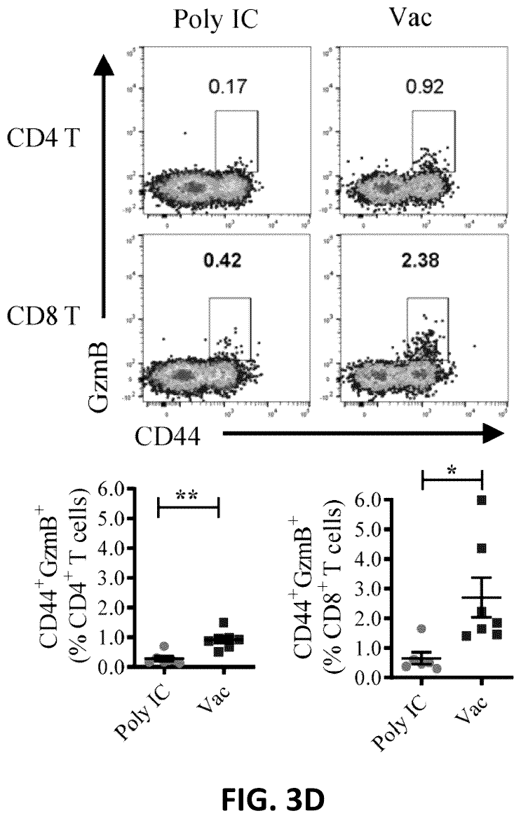

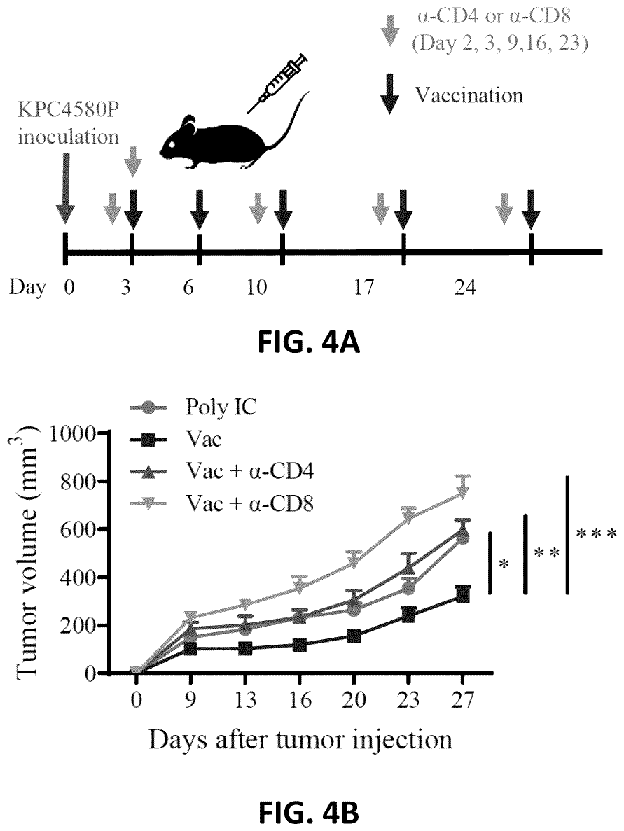

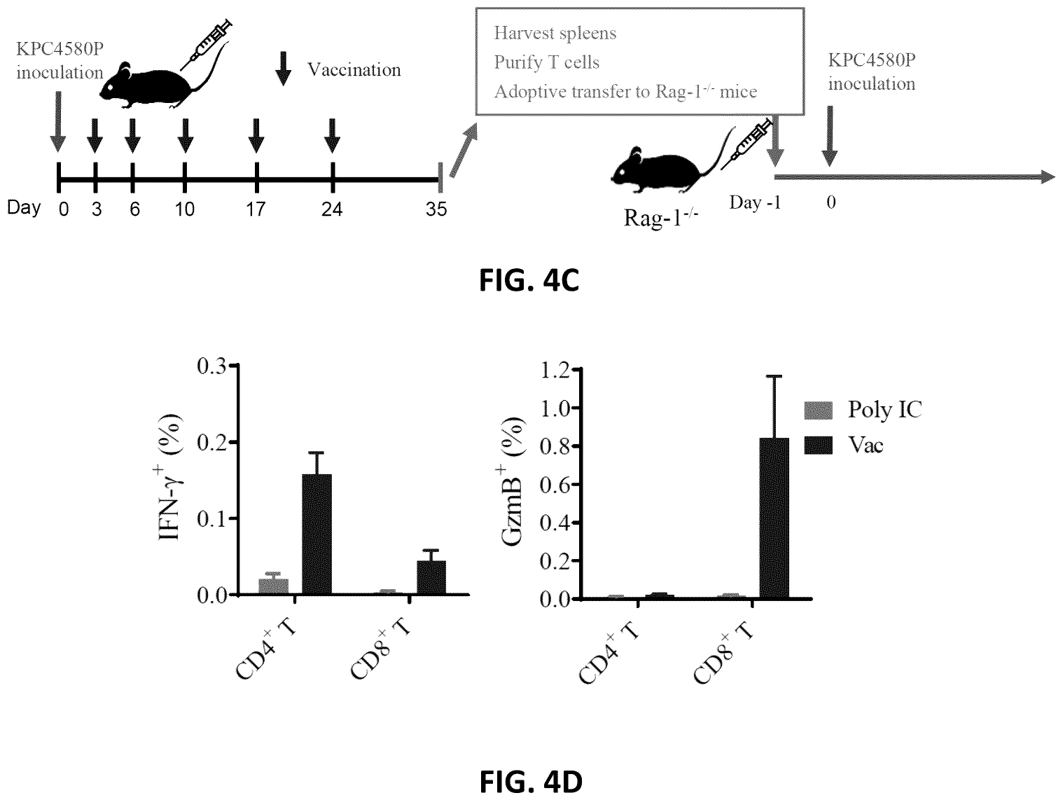

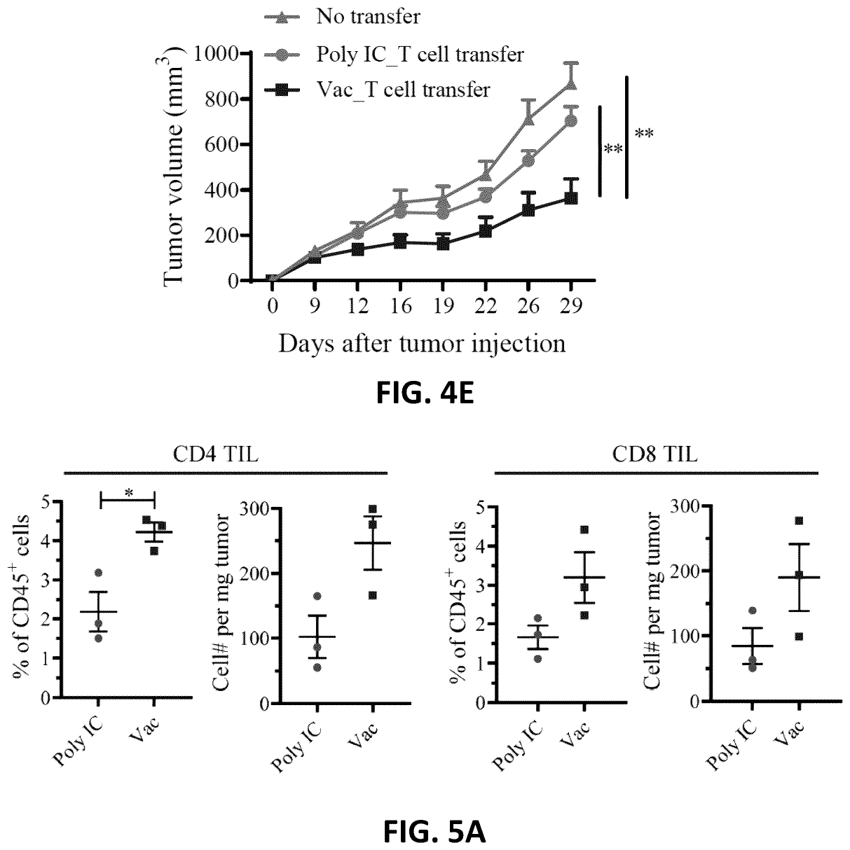

Neoantigen SLP vaccine induces neoantigen-specific CD4 and CD8 T cell responses capable of inhibiting KPC4580P growth. To test whether neoantigen-specific T cell responses generated by the mCAR12/mCDK12 neoantigen SLP vaccine protects mice from KPC4580P tumor challenge, mice were inoculated with tumor cells followed by mCAR12/mCDK12 SLP vaccination (FIG. 3A). Indeed, the neoantigen SLP vaccine (Vac) inhibited tumor growth (FIG. 3B). Vaccination was associated with robust mCAR12/mCDK12-specific CD4 T cell responses (FIG. 3C), and an increase in the number of splenic CD8 and CD4 T cells in vaccinated mice expressed cytotoxic marker GzmB (FIG. 3D). Depletion of T cells in vivo resulted in the abolishment of tumor protection (CD4 T cell depleted) or even enhanced tumor growth (CD8 T cell depleted) in vaccinated animals (FIG. 4A and FIG. 4B), indicating that both CD4 and CD8 T cells contribute to the antitumor immunity induced by neoantigen vaccination. To further validate the role of T cells induced by neoantigen vaccines in antitumor immune response, splenic T cells were isolated from vaccinated tumor-bearing mice and adoptively transferred into immunocompromised Rag-1^{-/-} mice, which lack mature T and B lymphocytes, followed by tumor challenge (FIG. 4C). The presence of neoantigen-specific T cells was confirmed before transfer by staining for intracellular IFN-γ and GzmB after ex vivo stimulation with mCAR12/mCDK12 peptides (FIG. 4D). Tumor growth in Rag-1^{-/-} mice demonstrated a significant reduction in tumor size only when the transferred T cells were obtained from vaccinated mice (FIG. 4E).

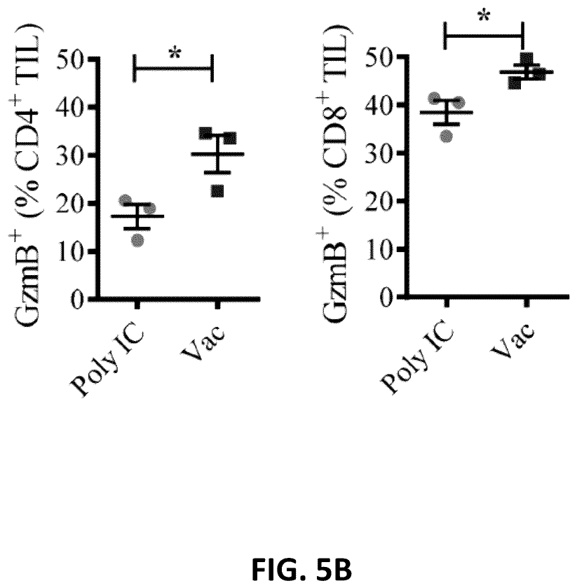

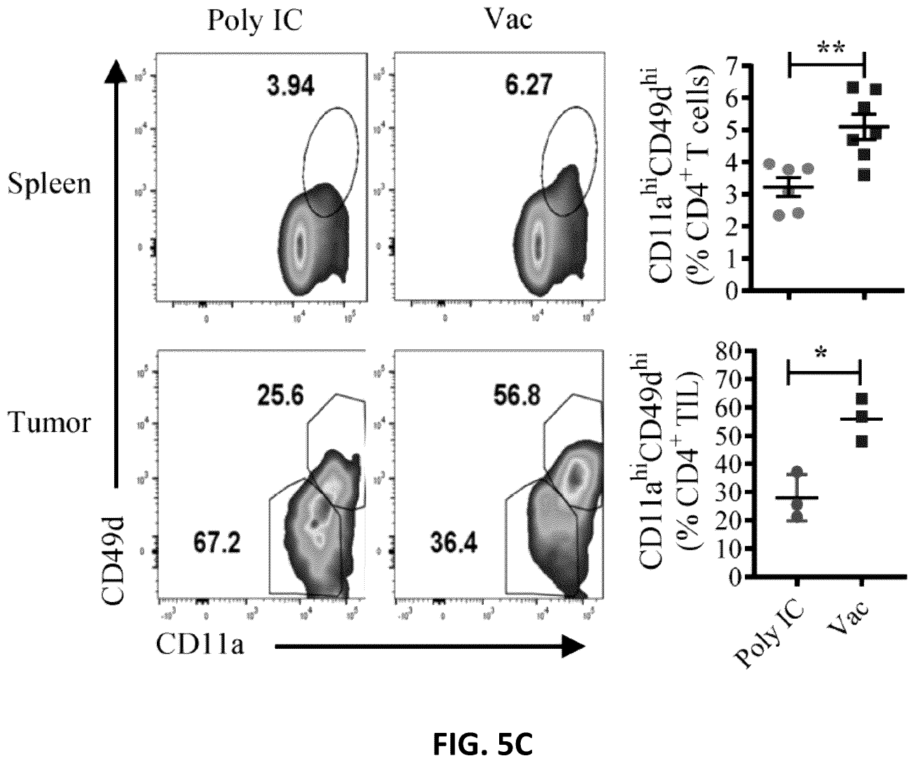

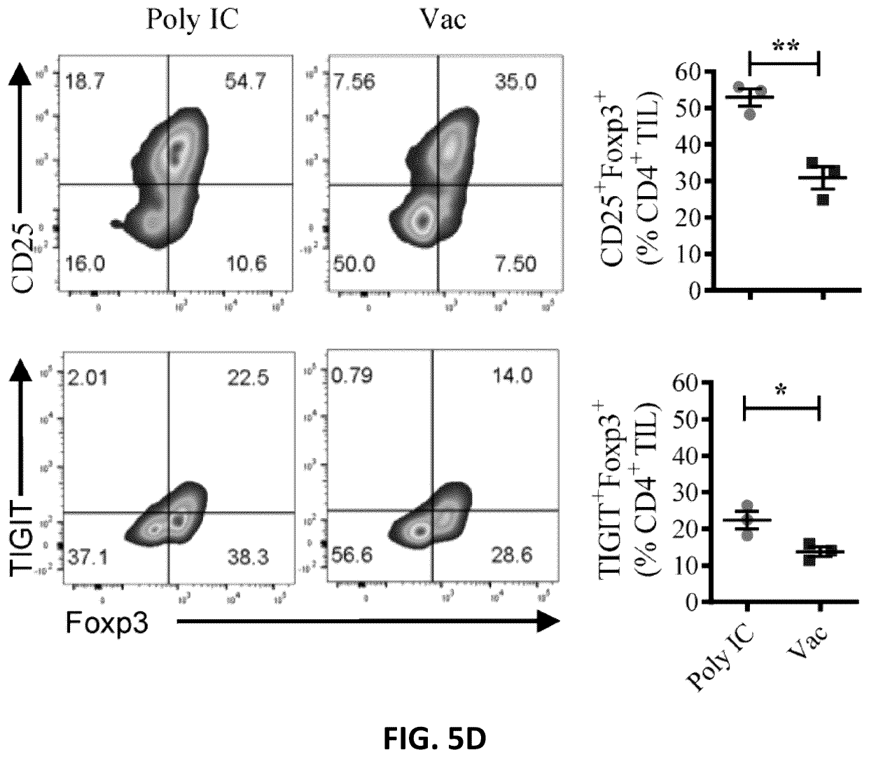

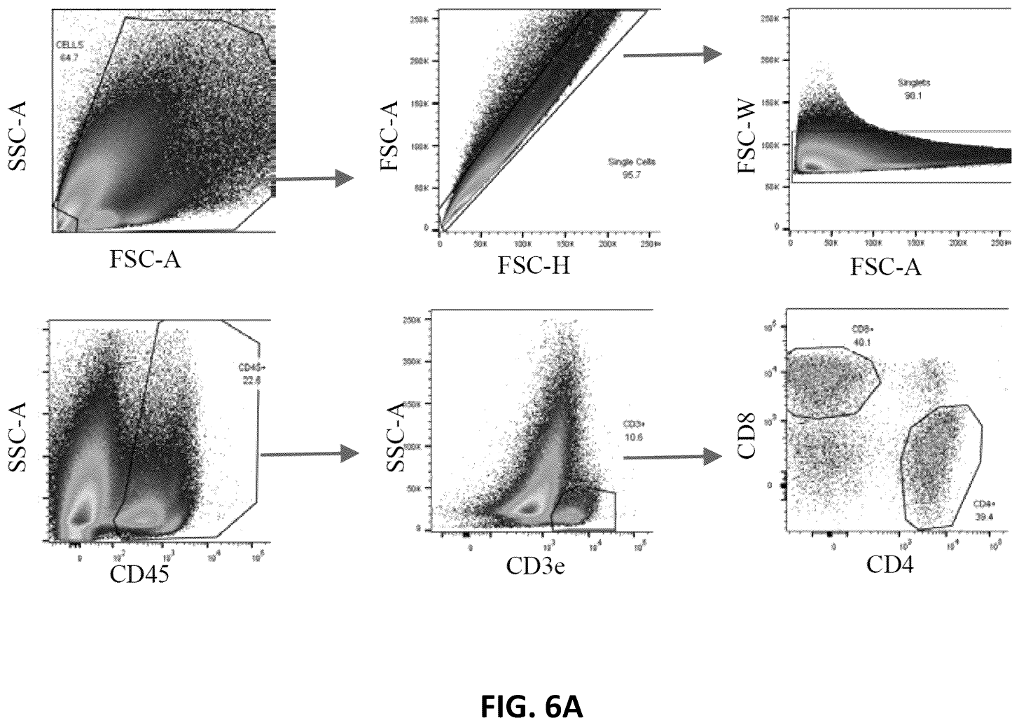

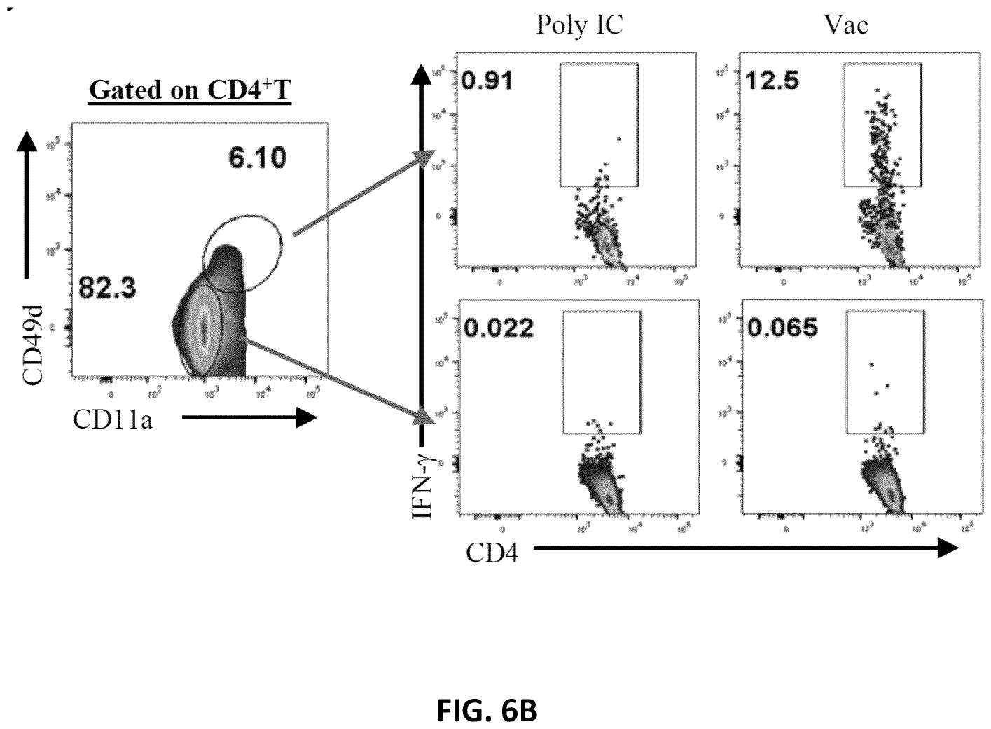

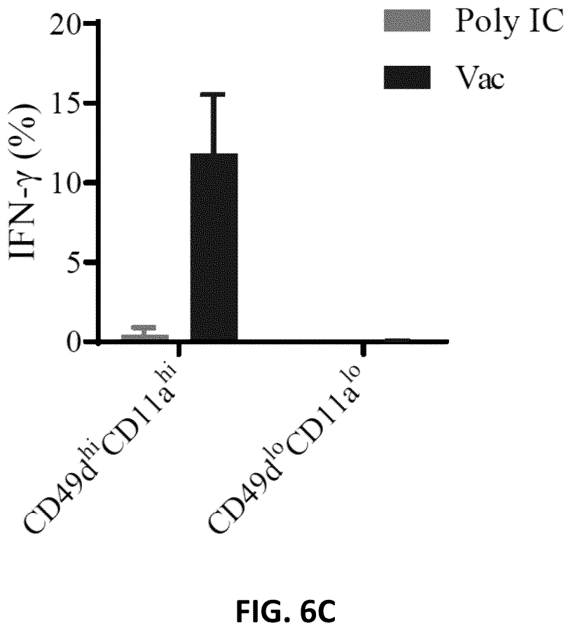

Neoantigen vaccine increases the number of functional tumor-specific CD4 T cells in the tumor microenvironment. Next, the effect of neoantigen vaccination on T cells in the tumor microenvironment was investigated. Tumors in vaccinated mice contained more infiltrating CD4 (4.22±0.42% vs 2.19±0.88%) and CD8 (3.2±1.12% vs 1.66±0.52%) T cells compared to unvaccinated tumors (FIG. 5A). GzmB expression was also detected in higher percentages of CD4 and CD8 tumor infiltrating lymphocytes (TILs) in vaccinated mice (FIG. 5B). Cell surface expression of integrin CD11a and CD49d was chosen as surrogate activation markers for antigen-experienced T cells. This approach based on the upregulation of CD49d and CD11a has proven valuable in identifying CD4 and CD8 T cells responding to human vaccines, in particular when there is limited information about the MHC restriction of epitopes/antigens. In addition, CD11a also appears to be a useful early activation marker for tumor-specific T cells. Both spleen cells and TILs harvested from KPC4580P tumor-bearing mice were stained. In mice vaccinated with neoantigens, compared to mice treated with poly IC alone, a greater percentage of CD4 T cells expressed high levels of CD11a and CD49d, both in spleen and in tumor (FIG. 5C). Representative gating scheme for CD4 and CD8 TILs is presented in FIG. 6A. Only the CD11a^{hi}CD49d^{hi}CD4 T cells, but not the CD11a^{lo}CD49d^{lo} subset, produced IFN-γ when stimulated with mCAR12/mCDK12 peptides in vitro (FIG. 6B, FIG. 6C), suggesting that CD11a^{hi}CD49d^{hi} T cells represent an antigen-experienced subpopulation in the KPC4580P tumor. Taken together, these data demonstrated that neoantigen vaccines result in more tumor-specific T cells with an activated/effector phenotype in the KPC4580P TME.

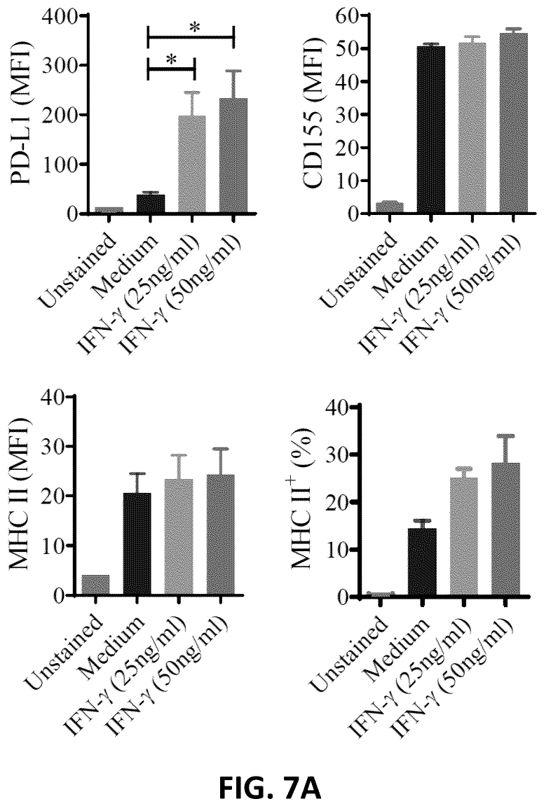

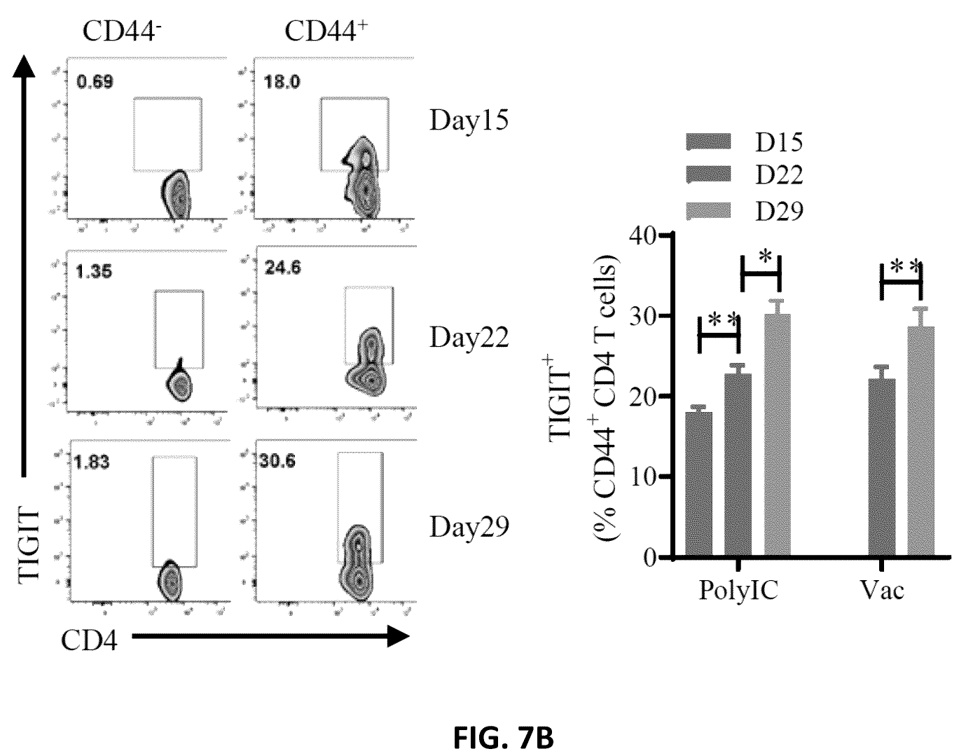

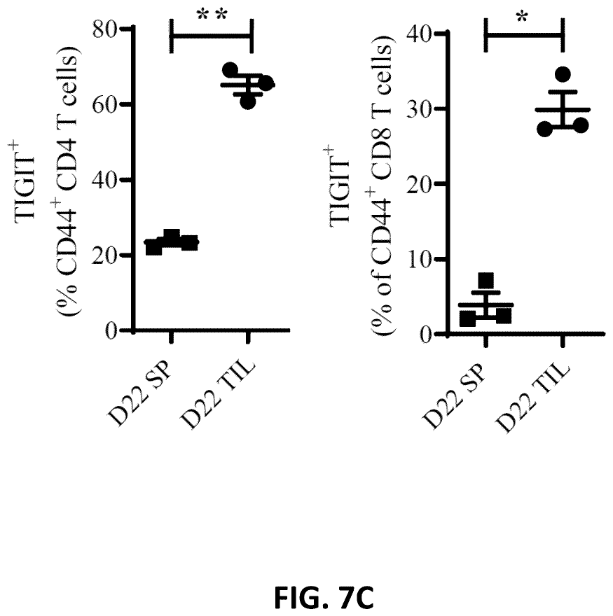

Evidence that TIGIT signaling is capable of inducing T cell exhaustion in the KPC4580P tumor model. The inability to completely reject KPC4580P tumors despite the enhanced tumor-specific T cell responses induced by the neoantigen vaccine led to investigation of potential immune checkpoints. Recently studies have identified a novel CD155/TIGIT axis of inhibition in both murine and human PDAC, and dual TIGIT and PD-1 blockade plus CD40 agonist stimulation was shown to be able to overcome T cell dysfunction in responder mice with established PDAC. Therefore, the role of TIGIT was investigated in mice challenged with KPC4580P tumors, which express both PD-L1 and the TIGIT ligand CD155, as well as low level MHC class II (FIG. 7A). In KPC4580P tumor bearing mice, TIGIT⁺ T cells were present in spleens and were enriched in the TILs (FIG. 7B and FIG. 7C), indicating a T cell exhaustion/dysfunctional phenotype. Of note, TIGIT expression was limited to the CD44⁺ memory subset and the expression level increased over time during tumor development (FIG. 7B). Neoantigen vaccination was associated with a decrease in the percentage of regulatory T cells (Treg, CD4⁺CD25⁺FoxP3⁺) and, in particular, TIGIT⁺ Treg in the tumor (FIG. 5D) likely due to the increased absolute number of tumor-infiltrating CD4 T cells (FIG. 5A). Although the mechanisms that lead to the relative reduction in Treg frequency in the tumor are unknown, this finding is consistent with other studies that demonstrated a decrease in Treg percentage after neoantigen vaccination.

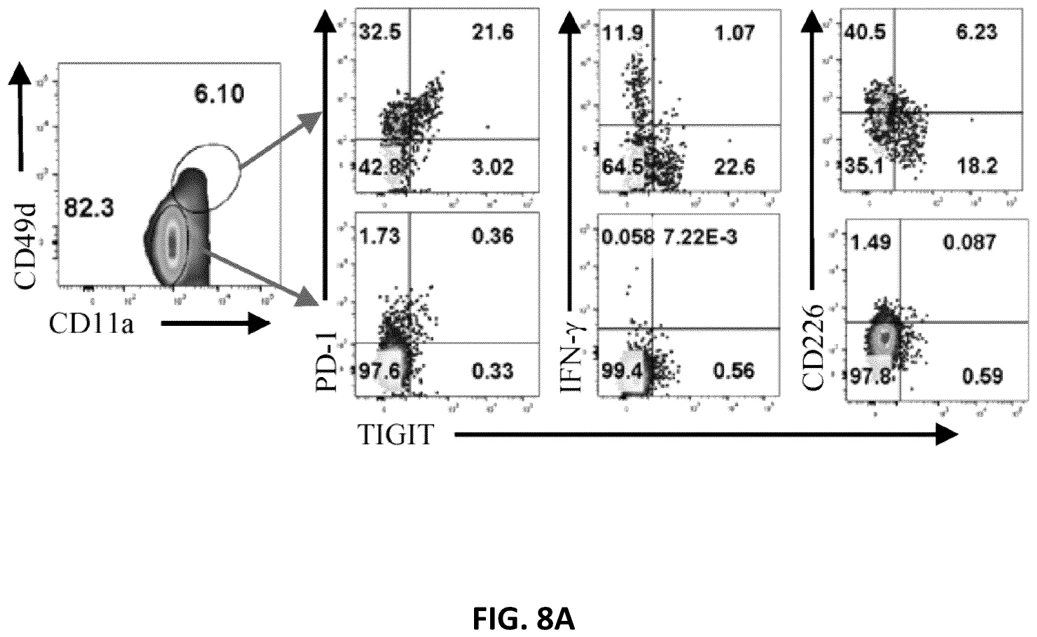

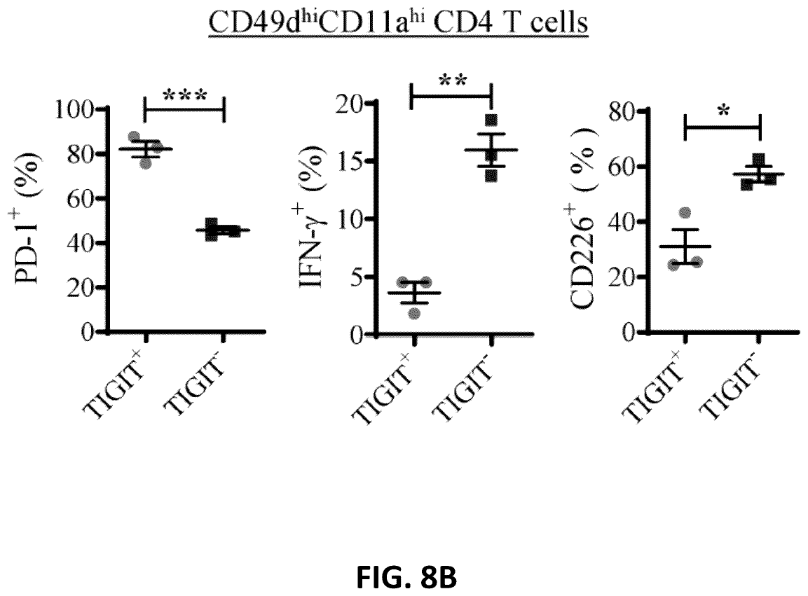

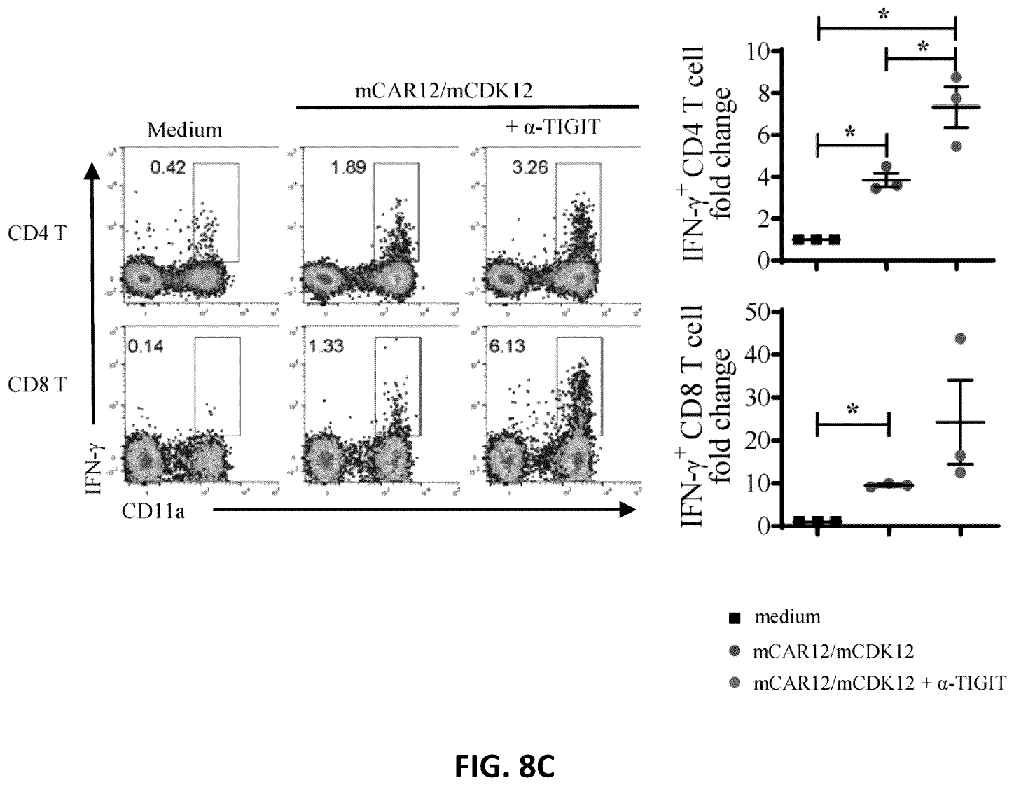

Studies have shown that TIGIT signaling inhibits T cell activation, cytokine production and TCR-mediated T cell proliferation. It was investigated herein whether TIGIT blockade reverses TIGIT-mediated exhaustion of neoantigen-specific T cells in response to peptide re-stimulation. In the spleens of KPC4580P tumor bearing mice, the TIGIT⁺ CD4 T cells were mostly found in the antigen-experienced CD11a^{hi}CD49d^{hi} cell population (FIG. 8A) and did not produce IFN-γ after in vitro re-stimulation (FIG. 8A and FIG. 8B). However, when spleen cells from vaccinated tumor bearing mice were stimulated with mCAR12/mCDK12 in the presence of the anti-TIGIT antagonist antibody, more CD4 and CD8 T cells produced IFN-γ as assessed by flow cytometry (FIG. 8C). These results demonstrate that TIGIT blockade is able to re-activate dysfunctional neoantigen-specific T cells and support the combination of neoantigen vaccine and TIGIT blockade in the treatment of pancreatic cancers.

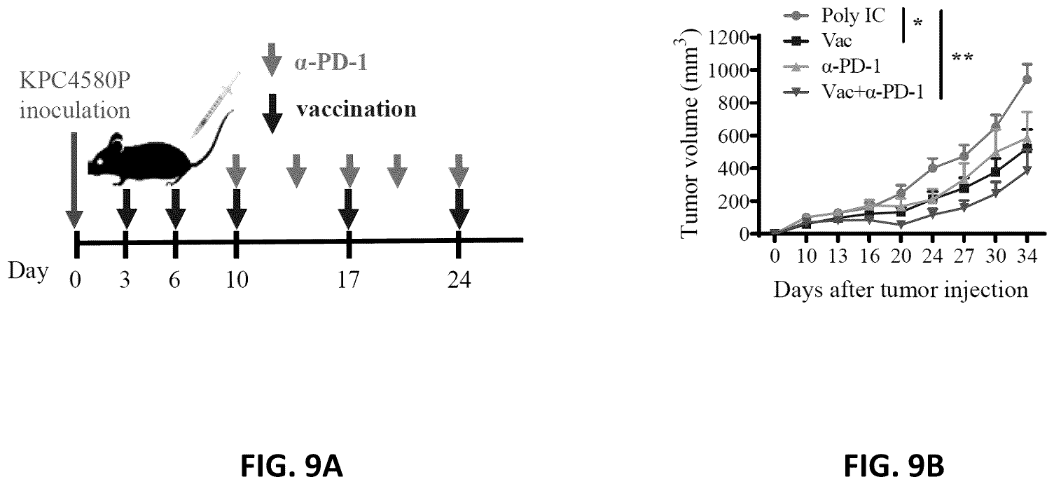

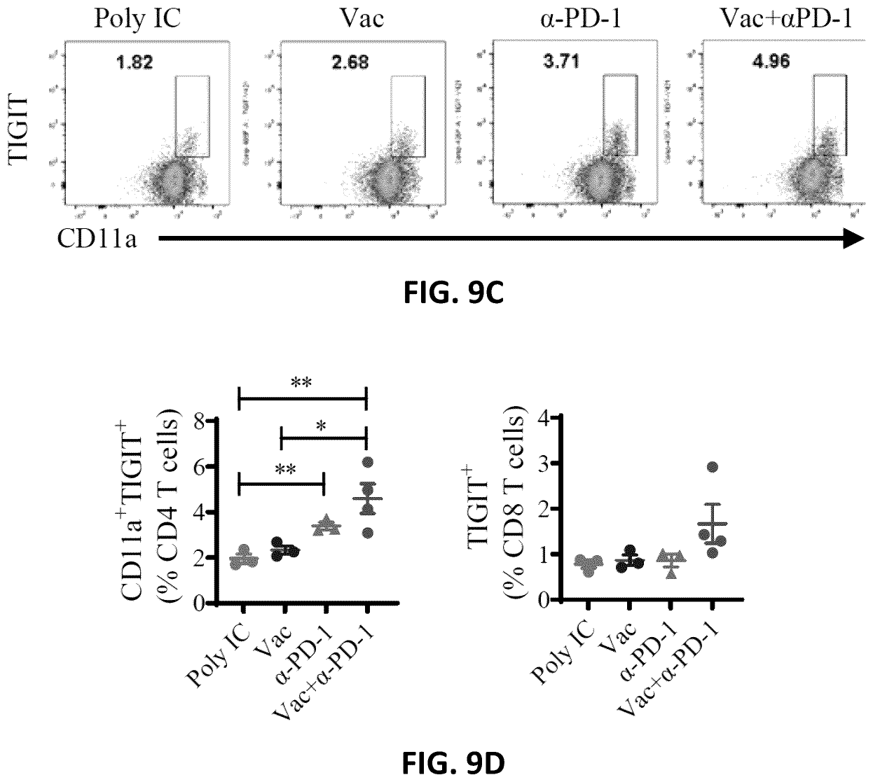

Combination TIGIT/PD-1 blockade enhances the ability of neoantigen vaccines to induce antitumor immunity. In mouse tumors, dysfunctional T cells were found to co-express TIGIT and PD-1, and dual blockade of TIGIT and PD-1 signaling pathways has synergistic effects on intra-tumoral T cells. Similarly, it was found that in KPC4580P tumor bearing mice, the majority (80%) of the TIGIT⁺ cells also express PD-1 but low levels of CD226 (FIG. 8A and FIG. 8B). Combining neoantigen vaccine with only anti-PD-1 treatment modestly enhanced KPC4580P tumor protection (FIG. 9A and FIG. 9B). Additional analyses indicated that anti-PD-1 treatment resulted in an increase of TIGIT expression in CD4 T cells and CD8 T cells (FIG. 9C and FIG. 9D). As described herein, dual blockade of PD-1 and TIGIT will synergize with neoantigen vaccination in generating optimal anti-tumor immune response in the KPC4580P pancreatic cancer model.

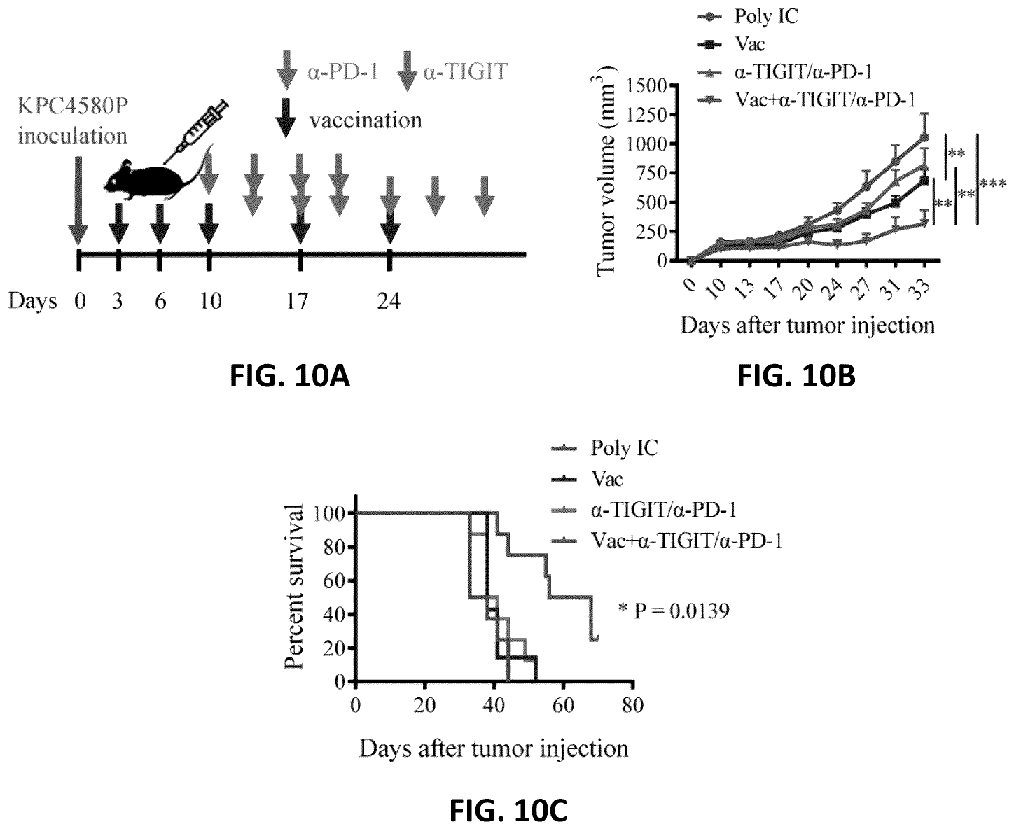

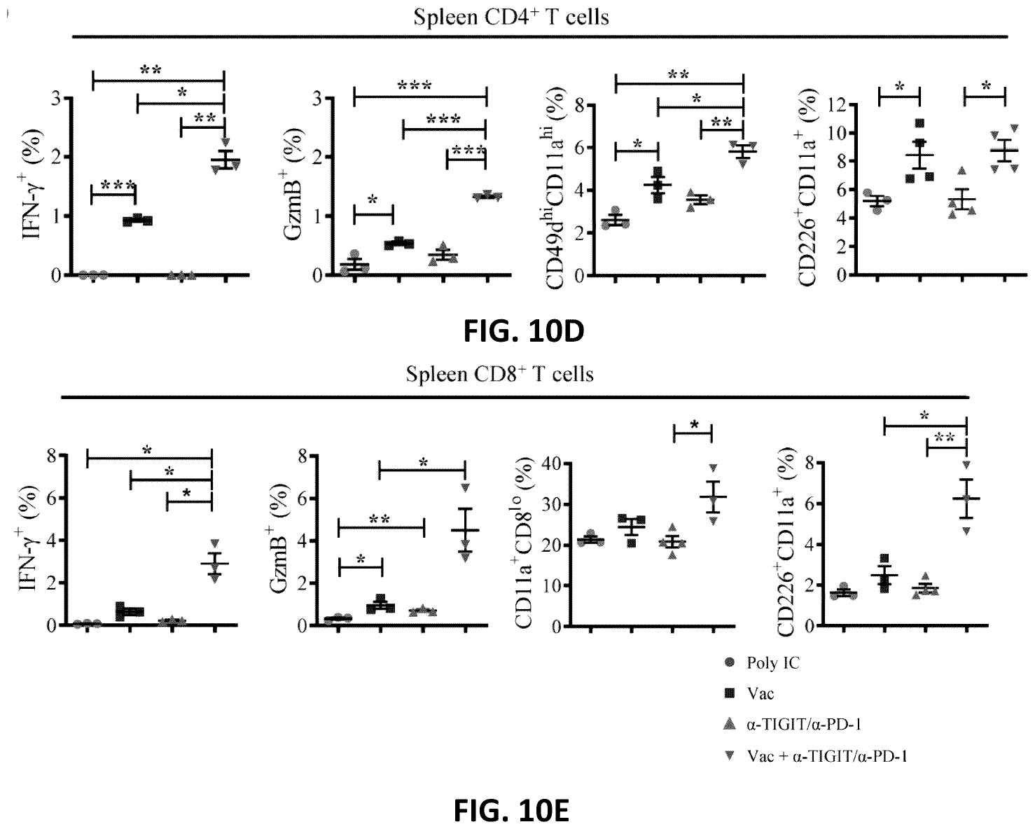

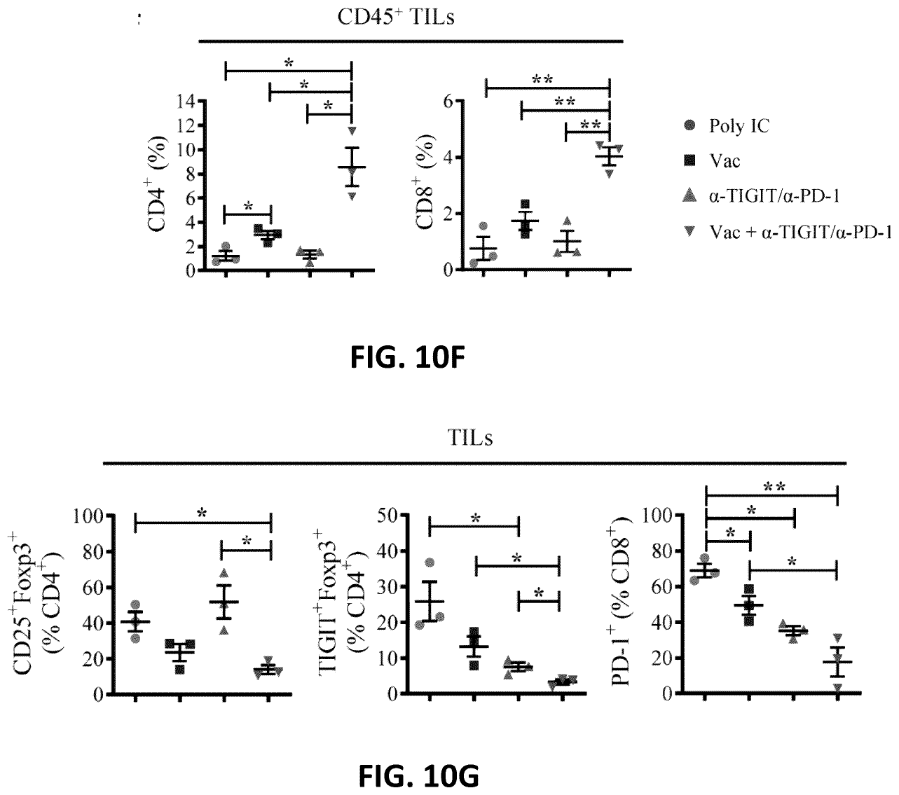

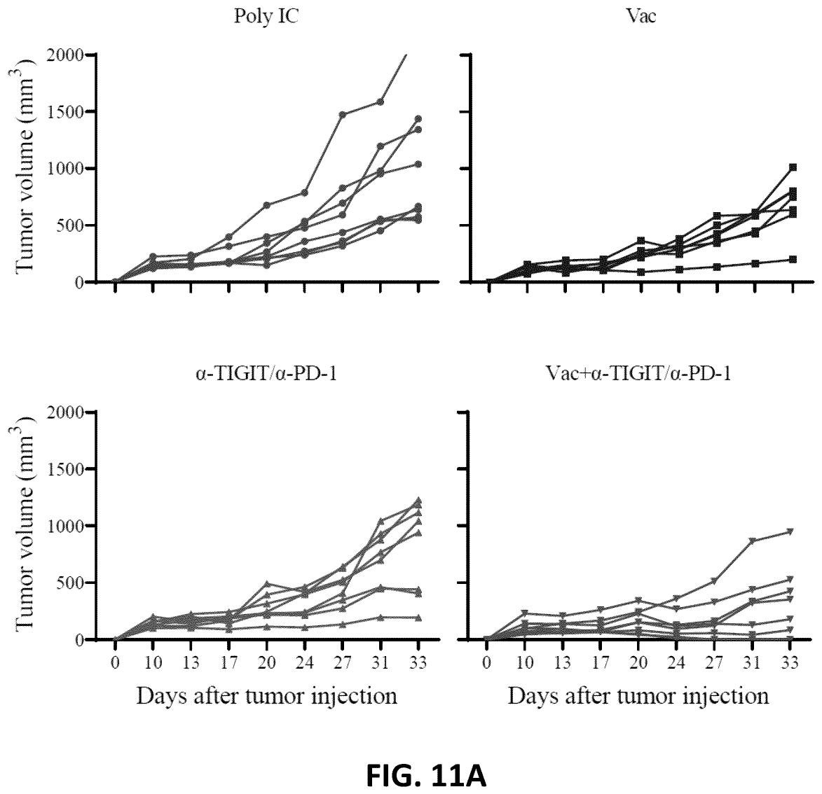

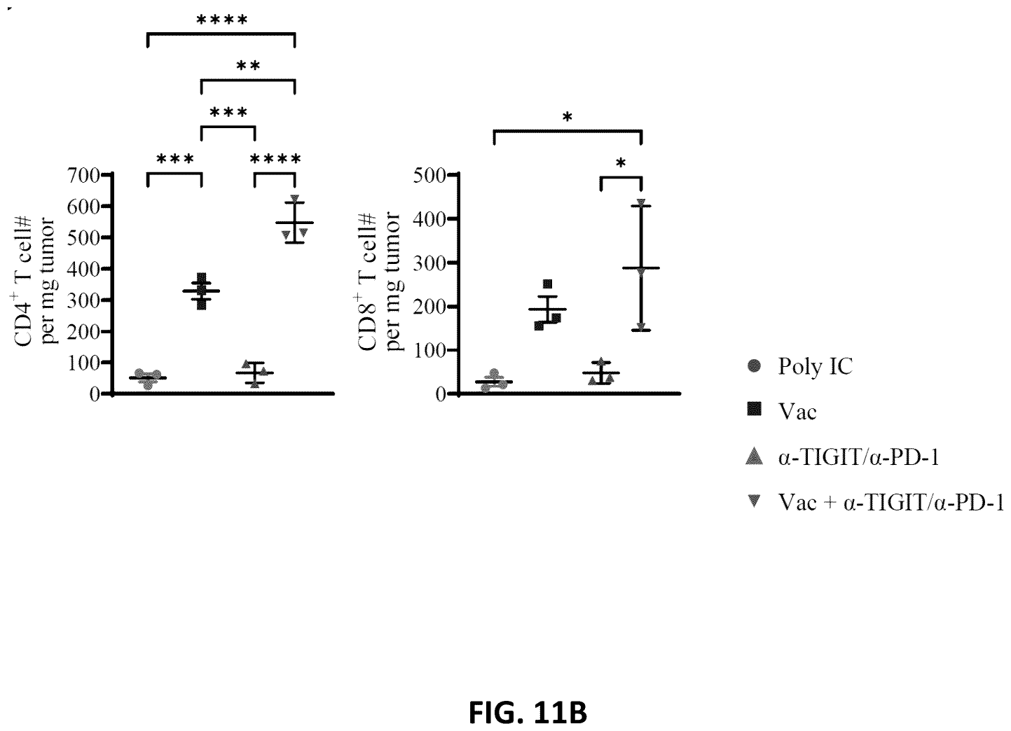

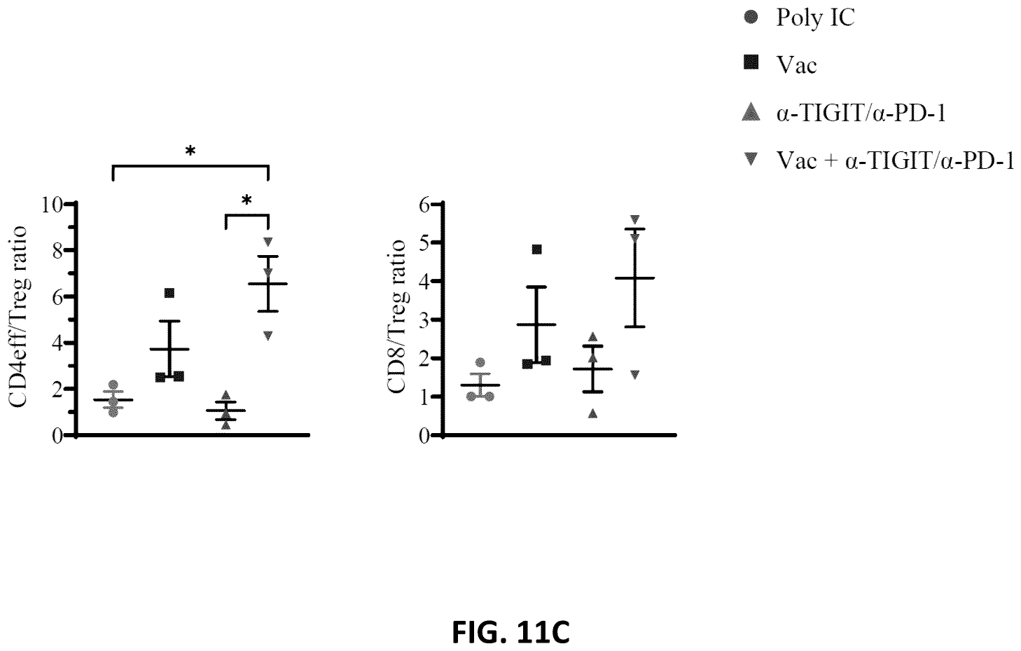

C57BL/6 were inoculated with KPC4580 cells at day 0 followed by vaccination starting at day 3. Treatments with anti-TIGIT and anti-PD-1 started at day 10 and day 13, respectively (see FIG. 10A for detailed treatment schema). Tumor sizes were measured twice weekly. Although TIGIT and PD-1 dual blockade alone did not seem to impact tumor growth, combining neoantigen vaccine substantially suppressed tumor growth (FIG. 10B and FIG. 11A) and led to longer survival of tumor-bearing animals (FIG. 10C). In addition, combination therapy also had a significant impact on the number and phenotype of neoantigen-specific T cells in the spleen and tumor microenvironment. Compared to vaccine alone, combination therapy resulted in higher percentage of splenic CD4 and CD8 T cells that produce IFN-γ and GzmB in response to neoantigen re-stimulation (FIG. 10D and FIG. 10E). The frequency of effector splenic CD4 T cells (CD11a^{hi}CD49d^{hi}, CD226⁺) also increased in mice receiving combination therapy (FIG. 10D). It is worth noting that although neoantigen vaccination alone did not induce a robust CD8 T cell response, dual TIGIT/PD-1 blockade was able to significantly enhance the percentage of CD226+CD8 T cells and neoantigen-specific CD8 T cell response (FIG. 10E). There were also more CD4 and CD8 TILs in tumors treated with combination therapy as compared to those treated with either neoantigen vaccine or anti-TIGIT/anti-PD-1 antibodies alone (FIG. 10F and FIG. 11B). Even though all CD4 T cell subsets increase following vaccination including Tregs when normalized using cell count per mg tumor (data not shown), some T cell subsets expand much more than others. The CD4eff/Treg and CD8/Treg ratios increased following neoantigen vaccination (FIG. 11C). As a result, there were lower percentages of Tregs (in particular, TIGIT+ Tregs) and PD-1+CD8 T cells in the tumors treated with combination therapy (FIG. 10G). These data demonstrate that dual PD-1/TIGIT blockade enhances immune responses induced by neoantigen vaccine, which results in superior antitumor immunity.

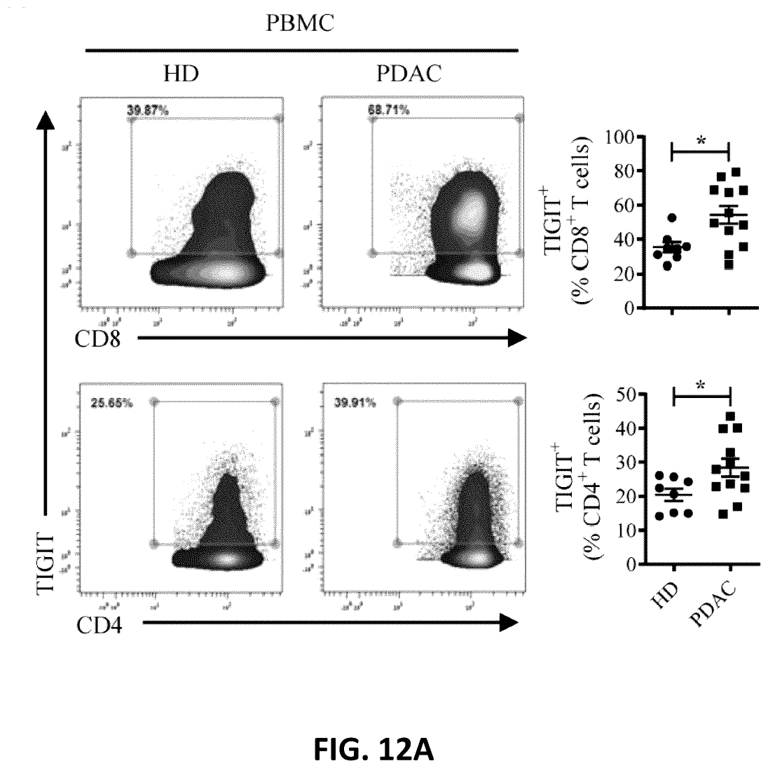

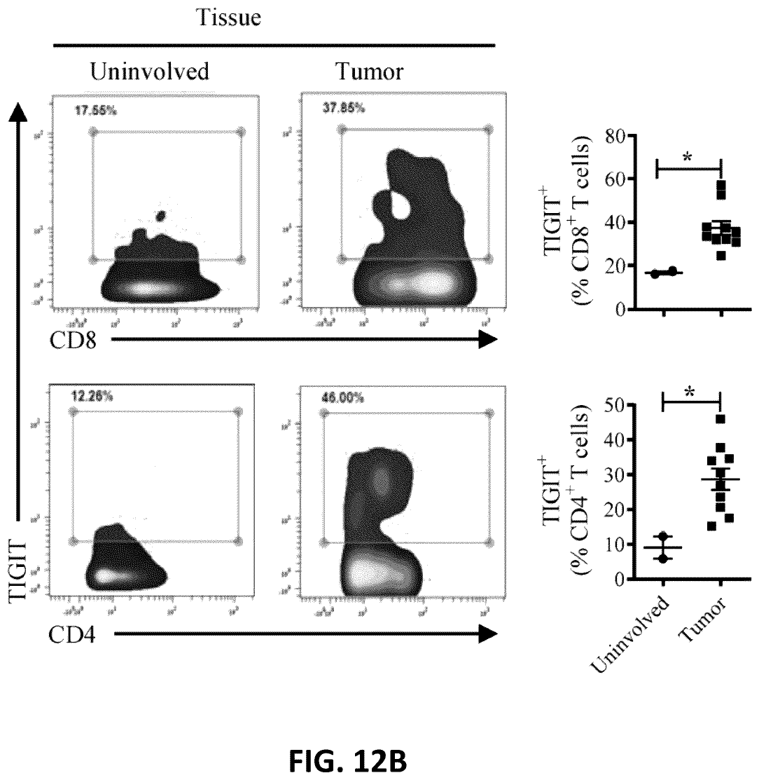

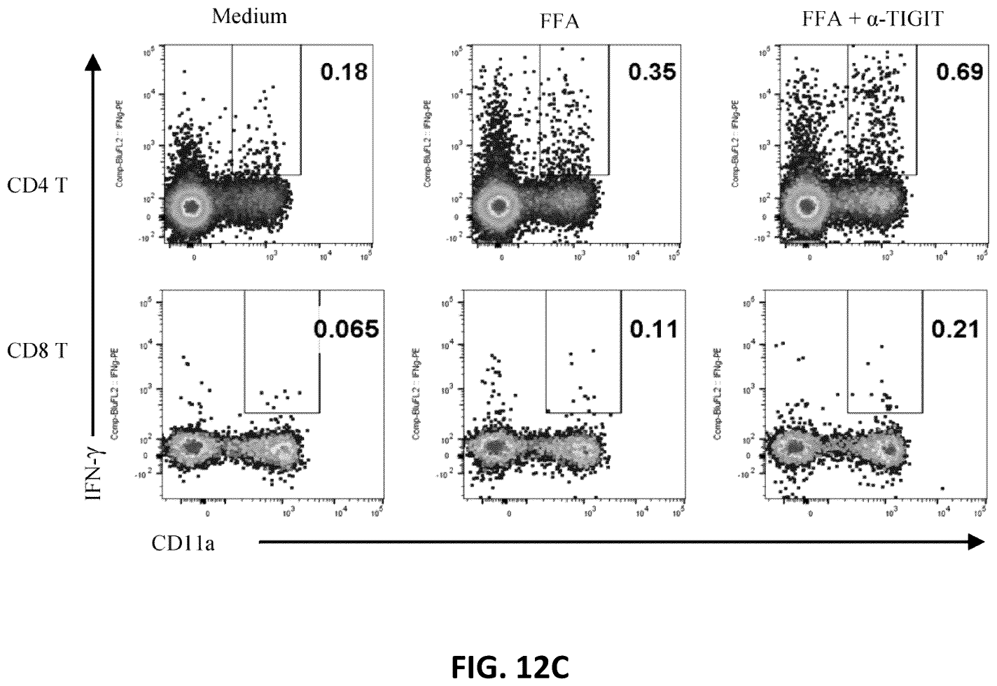

TIGIT expression and evidence of TIGIT signaling in patients with pancreatic cancer. To extend these findings, it was investigated herein whether TIGIT signaling is an important immune regulatory pathway in human pancreatic cancer. To do this, the expression of TIGIT was first examined in peripheral blood and tumor specimens derived from PDAC patients. CyTOF analyses indicated that TIGIT expression is increased on peripheral CD4 and CD8 T cells in human PDAC (FIG. 12A). TIGIT expression was also compared in T cells isolated from human PDAC (n=10) and a limited number (n=2) of adjacent uninvolved tissue and a significantly higher TIGIT expression was found in tumor tissues than in uninvolved adjacent tissues (FIG. 12B). These findings are in agreement with a recent report that human pancreatic cancer has an increased TIGIT protein expression on T and NK cells.

To test whether TIGIT signaling blockade can reinvigorate T cell responses in patients with pancreatic cancer, anti-TIGIT Ab was added to in vitro T cell cultures using PBMCs from a pancreatic cancer patient treated with a polyepitope neoantigen DNA vaccine on an expanded access protocol. The DNA vaccine was constructed as described previously and was manufactured in the GMP facility at WUSM. The neoantigen DNA vaccine was administered monthly using an integrated electroporation device. A total of 14 neoantigens were targeted, and neoantigen-specific T cell responses were detected against three neoantigens (FOXP3 (p.A349T), FAM129C (p.G520R), and ANK2 (p.R2714H). To determine whether the TIGIT blockade is capable of reversing any potential neoantigen-specific T cell exhaustion, post-vaccine PBMCs were stimulated with a mix of the three neoantigen peptides plus IL-2 for 3 days with or without the anti-TIGIT antibody. The cells were rested for 3 days followed by peptide re-stimulation and analyzed by intracellular cytokine staining and flow cytometry (FIG. 12C). The results show a roughly 2-fold increase in IFN-γ producing CD4 and CD8 T cells when anti-TIGIT antibody was included in the initial 3-day culture. Although a single patient sample was tested herein, these data suggest that blocking TIGIT signaling has the potential to reverse T cell dysfunction and provide support for further investigation of the combination of neoantigen vaccine and anti-TIGIT immunotherapy in human PDAC patients.

### DISCUSSION

Neoantigens have been demonstrated to drive potent anti-tumor T cell responses. Herein, a neoantigen vaccine was generated which comprised two 20-mer SLPs identified in the KPC4580P pancreatic cancer model. The neoantigen SLP vaccine was able to induce neoantigen-specific T cells in mice and reduce tumor growth. In combination with PD-1/TIGIT blockade, neoantigen vaccination resulted in enhanced tumor regression. The present disclosure provides support for combination therapy using neoantigen vaccines plus immune checkpoint inhibition targeting PD-1/TIGIT in pancreatic cancer patients.

Recent studies in three preclinical tumor models indicated that CD4 T cells play an important role in tumor control. The findings here are in line with these studies; the two neoantigens herein elicited predominantly CD4 T cells. The model herein provides potential insights into the function of neoantigen-specific CD4 T cells. The surrogate activation markers CD11a and CD49d were used to assess the T cell responses in tumors.

Expression of CD11a was initially used to track antigen-primed effector and memory T cells induced by viral vaccination, but more recently, it has been demonstrated that high expression of CD11a can also be used as a marker to identify and track endogenous tumor reactive CD8 T cells. Herein, neoantigen vaccinated tumor-bearing mice display more CD11a^{hi}CD49d^{hi}CD4 T cells and lower percentage of Tregs in the TME compared to vehicle-treated tumor-bearing mice. The CD11a^{hi}CD49d^{hi} CD4 T cells in vaccinated mice comprised the majority of IFN-γ- and GzmB-producing cells (FIG. 10A-FIG. 10G). Of note, the CD11a^{hi}CD49d^{lo} T cell population has not been examined for its functionality and neoantigen-specificity. It is possible that the CD8 T cell response induced following vaccination may not be entirely mCAR12 and mCDK12 specific. A recent study using a Plasmodium infection model indicated that activated CD4 T cells develop into both CD11a^{hi}CD49d^{hi} type 1 helper T (Th1) cells and CD11a^{hi}CD49d^{lo} follicular helper T (Tfh)-like cells. The exact mechanism through which neoantigen-specific CD4 T cells mediate tumor regression is unknown at this point. The elevated level of GzmB in CD4 TIL compared to CD4 splenocytes indicates cytolytic function. It is possible that CD4 TIL can directly mediate tumor cell killing. At baseline, a small percentage (14.4%) of KPC4580P tumor cells express low levels of MHC class II. Upon IFN-γ treatment, the percentage of KPC4580P cells that express MHC II increases to 25.2-28.2% (FIG. 7A). Further studies, perhaps through a modification of the adoptive T cell transfer experiment in tumor-bearing Rag−/− mice (FIG. 4A-FIG. 4E) using CD4 T cells from perforin/GzmB knockout mice could provide further details on the exact mechanism. Given the dependence on CD8 T cells for tumor control (FIG. 4A-FIG. 4E), the data herein overall suggest that neoantigen vaccination induces specific CD4 T cells, and expands, and broadens the tumor-directed T cell response including neoantigen-specific CD8 T cells. However, the CD8 response induced by neoantigen vaccination is likely restrained by the upregulation of immune checkpoint molecules such as PD-1 and TIGIT. It was demonstrated herein that dual blockade of TIGIT and PD-1 can enhance the CD8 T cell response to neoantigen vaccines. Of note, neoantigen-specific CD4 T cells have been identified in several neoantigen vaccine studies including a neoantigen DNA vaccine trial in TNBC. Neoantigen-reactive CD4 T cells have also been shown to mediate clinical regression in a patient with cholangiocarcinoma when neoantigen-reactive CD4 T cells were adoptively transferred, further confirming the important contribution of neoantigen-specific CD4 T cells towards antitumor immunity.

As has been described in multiple reports, intratumoral CD8 T cells in PDAC display an exhausted phenotype, typified by the expression of TIGIT and frequently of PD-1. The data herein extend these findings, demonstrating that TIGIT⁺ CD4 T cells express higher levels of PD-1, less CD226, and produce less IFN-γ than TIGIT− CD4 T cells (FIG. 8A-FIG. 8B), suggesting a dysfunctional phenotype of the TIGIT+CD4 T cells. While neoantigen vaccination or TIGIT blockade partially restored immune function, this study also suggested that neoantigen vaccination (and possibly anti-TIGIT blockade) could also result in increased expression of the PD-1/PD-L1 pathway (FIG. 8C and FIG. 7A-FIG. 7C), possibly through activated effector T cells producing IFN-γ. Indeed, the data showed that exposure of KPC4580P tumor cells to IFN-γ greatly increased PD-L1 expression, with the potential to bind to PD-1 on neoantigen activated T cells leading to T cell dysfunction. The data also show that combination therapy of neoantigen vaccine plus anti-PD-1 modestly enhanced tumor protection which may be related to the observation that PD-1 treatment increased TIGIT expression in T cells (FIG. 9C). This finding is consistent with a study on hepatocellular cancer showing anti-PD-1 therapy greatly upregulated TIGIT expression in activated T cells and the CD155/TIGIT axis contributed to anti-PD-1 treatment resistance. Additionally, recent studies demonstrated that the CD155/TIGIT axis is a key driver of immune evasion in pancreas cancer, and that both PD-1 and TIGIT signaling impairs CD226 co-stimulation which is required for restoring antitumor immunity. Based on this, and the observation herein that CD226 is readily expressed on neoantigen-specific T cells after vaccination (FIG. 10D and FIG. 10E), tumor-bearing mice were treated with combination PD-1/TIGIT blockade and neoantigen vaccine. This combination not only improved vaccine-induced T cell responses, but also enhanced T cell infiltration in the tumor. Of note, combination PD-1/TIGIT blockade has entered clinical testing. In patients with NSCLC, combination therapy showed meaningful improvement in response rate and progression-free survival. However, combination PD-1/TIGIT blockade using the same antibodies plus chemotherapy did not meet the primary endpoints of progression-free survival and overall survival in patients with extensive-stage SCLC. It is likely that the benefit of combination PD-1/TIGIT therapy will be dependent on the cancer type and clinical context. In this context, TIGIT blockade appears highly relevant in patients with PDAC. It was previously shown that TIGIT expression is increased on T and NK cells in pancreatic cancer and its expression in the tumors correlates with its expression in matched blood. The CyTOF data herein showed TIGIT expression is increased in both CD4 and CD8 T cells compared to healthy donors and higher TIGIT expression was found on immune cells from PDAC tumors compared to uninvolved tissue. The present study suggests that targeting the PD-1 and TIGIT signaling pathways enhances the response to neoantigen vaccines in pancreatic cancer, highlighting the potential synergy of these therapies in pancreatic cancer.

Accordingly, the present disclosure provides the first evidence that dual immune checkpoint PD-1/TIGIT blockade enhances therapeutic response to neoantigen vaccine. These findings have direct clinical implications for combination PD-1/TIGIT blockade and neoantigen vaccine enhancing the therapeutic efficacy of immunotherapy in pancreatic cancer patients.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) are construed to cover both the singular and the plural, unless specifically noted otherwise. In some embodiments, the term “or” as used herein, including the claims, is used to mean “and/or” unless explicitly indicated to refer to alternatives only or to refer to the alternatives that are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and may also cover other unlisted steps. Similarly, any composition or device that “comprises,” “has” or “includes” one or more features is not limited to possessing only those one or more features and may cover other unlisted features.

All methods described herein are performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

To facilitate the understanding of the embodiments described herein, a number of terms are defined below. The terms defined herein have meanings as commonly understood by a person of ordinary skill in the areas relevant to the present disclosure. Terms such as “a,” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of treating pancreatic cancer in a subject, the method comprising administering a therapeutically effective amount of a composition comprising:
a neoantigen vaccine comprising at least one pancreatic cancer-associated neoantigen; and
at least one immune checkpoint inhibitor.

2. The method of claim 1, wherein the at least one immune checkpoint inhibitor comprises at least one of a PD-1 inhibitor, a PD-1L inhibitor, and a TIGIT inhibitor.

3. The method of claim 2, wherein the at least one immune checkpoint inhibitor comprises a PD-1 inhibitor and a TIGIT inhibitor.

4. The method of claim 3, wherein administering the therapeutically effective amount of the composition increases survival, enhances T cell antitumor immune response or infiltration, or reduces tumor volume in the subject compared to administering a neoantigen vaccine or checkpoint inhibitor alone.

5. The method of claim 2, wherein the at least one immune checkpoint inhibitor comprises at least one of an anti-PD1 antibody, an anti-PDL1 antibody, and an anti-TIGIT antibody.

6. The method of claim 1, wherein the at least one pancreatic cancer-associated neoantigen is identified based on at least one of exome sequencing and RNA sequencing of a pancreatic tumor or cancer cell.

7. The method of claim 1, wherein the at least one pancreatic cancer-associated neoantigen comprises at least a portion of a protein or peptide encoded by a gene selected from the group consisting of CAR12, CDK12, FOXP3, FAM129C, and ANK2.

8. The method of claim 1, wherein the at least one pancreatic cancer-associated neoantigen comprises at least one amino acid sequence, each amino acid sequence at least 95% identical to a sequence selected from the group consisting of SEQ ID NOS: 1-5.

9. The method of claim 1, wherein the at least one pancreatic cancer-associated neoantigen comprises at least one amino acid sequence, each amino acid sequence at least 95% identical to SEQ ID NO: 1 or SEQ ID NO: 2.

10. The method of claim 1, wherein the at least one pancreatic cancer-associated neoantigen comprises at least one amino acid sequence, each amino acid sequence at least 95% identical to a sequence selected from the group consisting of SEQ ID NOS: 7-12.

11. The method of claim 1, wherein the therapeutically effective amount of the composition induces a neoantigen-specific CD4 or CD8 T cell antitumor response.

12. The method of claim 1, wherein the therapeutically effective amount of the composition increases the number of functional tumor-specific CD4 T cells in a tumor microenvironment (TME) or spleen of the subject compared to administering a neoantigen vaccine or checkpoint inhibitor alone.

13. The method of claim 1, wherein the therapeutically effective amount of the composition reduces or prevents TIGIT-mediated exhaustion of neoantigen-specific T cells compared to administering a neoantigen vaccine or checkpoint inhibitor alone.

14. A pharmaceutical composition comprising a neoantigen vaccine, the neoantigen vaccine comprising at least one pancreatic cancer-associated neoantigen and at least one immune checkpoint inhibitor.

15. The composition of claim 14, wherein the at least one pancreatic cancer-associated neoantigen is derived from at least a portion of a protein or peptide encoded by a gene selected from the group consisting of CAR12, CDK12, FOXP3, FAM129C, and ANK2.

16. The composition of claim 14, wherein the at least one pancreatic cancer-associated neoantigen comprises at least one amino acid sequence, each amino acid sequence at least 95% identical to a sequence selected from the group consisting of SEQ ID NOS: 1-5.

17. The composition of claim 14, wherein the at least one pancreatic cancer-associated neoantigen comprises at least one amino acid sequence, each amino acid sequence at least 95% identical to a sequence selected from the group consisting of SEQ ID NOS: 7-12.

18. The composition of claim 14, wherein the at least one immune checkpoint inhibitor comprises at least one of a PD-1 inhibitor, a PD-1L inhibitor, and a TIGIT inhibitor.

19. The composition of claim 14, wherein the at least one immune checkpoint inhibitor comprises a PD-1 inhibitor and a TIGIT inhibitor.

20. A vaccine comprising a peptide comprising:
at least one pancreatic cancer-associated neoantigen amino acid sequence, wherein each pancreatic cancer-associated neoantigen amino acid sequence is at least 95% identical to a sequence selected from the group consisting of SEQ ID NOS: 1-5 and SEQ ID NOS: 7-12; and
a pharmaceutically acceptable carrier or adjuvant.
